(19) United States
(12) Patent Application Publication
(10) Pub. No.: US 2021/0403975 A1
(43) Pub. Date: Dec. 30, 2021

(54) PHENOTYPIC ANTIMICROBIAL SUSCEPTIBILITY PROFILING OF UNIDENTIFIED PATHOGENS DIRECTLY FROM PATIENT SAMPLES

(71) Applicant: GeneFluidics, Inc., Irwindale, CA (US)

(72) Inventors: Vincent Gau, Irwindale, CA (US); Michael Tomasek, Huntington Beach, CA (US); Jade Chen, Diamond Bar, CA (US)

(21) Appl. No.: 17/359,526

(22) Filed: Jun. 26, 2021

Related U.S. Application Data
(60) Provisional application No. 63/045,036, filed on Jun. 26, 2020.

(51) Int. Cl. C12Q 1/18; C12Q 1/02

(52) U.S. Cl. CPC C12Q 1/18; C12Q 1/025; G01N 2500/10; G01N 2458/00; G01N 2333/265

(57) ABSTRACT

The invention relates to methods for phenotypic antimicrobial susceptibility profiling of unidentified pathogens directly from patient samples and uses thereof, including providing timely important information for evidence-based management of patients with infections from unidentified pathogens.

### CROSS REFERENCE TO RELATED APPLICATIONS

This application claims priority under 35 U.S.C. § 119 from Provisional Application Ser. No. 63/045,036, filed Jun. 26, 2020, the disclosure of which is incorporated herein by reference.

### FIELD

The invention relates to methods for phenotypic antimicrobial susceptibility profiling of unidentified pathogens directly from patient samples, and uses thereof.

### BACKGROUND

Increasing global travel and changes in the environment may increase the frequency of contact with a natural host carrying an infection, and therefore increase our chances of encountering pathogens previously unknown to humans. During an emergency (man-made, natural disaster or pandemic), the etiology of infection might be unknown at the time of treating patients. The existing local or global Antimicrobial Stewardship Programs (ASP) might not be fully prepared for emerging/re-emerging infectious disease outbreaks, especially if they are caused by an unknown organism, engineered bioterrorist attack, or rapidly evolving superbug.

### SUMMARY

The disclosure provides for the development of a molecular test based on the transcriptional responses of causative bacteria directly in the specimen to antibiotic exposure. Quantification of species-specific 16S rRNA growth sequences are used to provide rapid AST results with >95% categorical agreement with reference AST methods according to CLSI guidelines M02 and M07, bypassing the necessity of overnight culture for generating isolates, pathogen identification or quantification of microbial load.

In a particular embodiment, the disclosure provides a method to assess the antimicrobial susceptibility profile of unidentified and/or unknown pathogens to a panel of antimicrobials directly from an unprocessed sample without the use of clinical isolates, comprising steps (i) or (ii); and (iii): (i) measuring from the unprocessed sample, a change in the level of a growth marker for the unidentified and/or unknown pathogens when the pathogens are exposed to the panel of antimicrobials; or (ii) measuring from the unprocessed sample, a change in the level of a growth marker for the unidentified and/or unknown pathogens when the unprocessed sample with unidentified and/or unknown pathogens are tested at a different level and exposed to the panel of antimicrobials at various antimicrobial/pathogen ratios; and (iii) comparing the change in the level of the growth marker for unidentified and/or unknown pathogen from steps (i) or (ii) to the level of the growth marker when the unidentified and/or unknown pathogens exposed to a growth control (GC) condition that lacks antimicrobials, wherein the susceptibility of the unidentified and/or unknown pathogens to the panel of antimicrobials is determined by a change in the level of the growth marker in comparison to the level of the growth marker when the unidentified and/or unknown pathogens are exposed to the growth control condition that lacks antimicrobials. In a further embodiment, the growth marker is selected from nucleic acids, proteins, and phenotypic characteristics. In yet a further embodiment, the growth marker is RNA, and wherein the change of RNA content is measured using one or more molecular analysis assays that utilize pathogen species-specific quantification, pathogen class-specific quantification, and/or universal quantification. In another embodiment, the pathogen species specific quantification includes specific quantification of Escherichia co/i, Klebsiella pneumoniae, and/or methicillin-resistant Staphylococcus aureus (MRSA); and wherein the pathogen class-specific quantification includes specific quantification of Enterobacteriaceae, Gram-negative bacteria, and/or Gram-positive bacteria. In yet another embodiment, the growth marker is RNA, and wherein the change of RNA content is measured using one or more molecular analysis assays that utilize enzymatic signal amplification with electrochemical sensors. In a further embodiment, the pathogens in the unprocessed sample are exposed to the panel of antimicrobials by using a macrodilution assay, a microdilution assay, by agar plating, or by culturing in growth media culture. In yet a further embodiment, defined set of concentrations for the panel of antimicrobials are used; or a range of concentrations for the panel of antimicrobials are used; or various antimicrobial-to-pathogen ratios are used; or the pathogens are exposed to the panel of antimicrobials using different exposure times. In a certain embodiment, the defined set of concentrations for the panel of antimicrobials includes concentrations that provide for susceptible, intermediate and/or resistance breakpoints. In another embodiment, the defined set of concentrations for the panel of antimicrobials includes concentrations that provide for a 2-fold increase or decrease from susceptible, intermediate and/or resistance breakpoints. In yet another embodiment, the defined set of concentrations for the panel of antimicrobials includes concentrations that provide for a more than 2-fold increase or decrease from susceptible, intermediate and/or resistance breakpoints. In a further embodiment, the defined set of concentrations for the panel of antimicrobials includes from 2-12 different concentrations for the panel of antimicrobials. In yet a further embodiment, pathogens cultured or diluted to different microbial levels are selected from viable pathogens cultured or diluted to a set number of different dilution levels, viable pathogens cultured or diluted within a range of microbial levels, and pathogens concentrated to different levels. In another embodiment, viable pathogens culture or diluted to different dilution levels includes pathogens the are (optionally multiplied through viability culture to 2×, 5×, 10×, 100× and/or 1000× depending upon initial concentration in the sample), diluted to 1×, 0.5×, 0.3×, 0.1×, 0.01×, 0.001×, 0.0001× and/or 0.00001×. In yet another embodiment, pathogens concentrated to different levels is by concentrating for different centrifugation times, by concentrating using different centrifugation forces, by different cycle number of pelleting using reagents (such as red blood cell lysis buffer) or culture medium to minimize matrix effect and/or by taking up the pathogen pellet in different volumes. In a further embodiment, the panel of antimicrobials includes 2, 3, 4, 5, 6, 7, 8, 9 or 10 antimicrobials. In yet a further embodiment, the panel of antimicrobials comprises ciprofloxacin, gentamicin, and/or meropenem. In another embodiment, the method is used to assess in an unprocessed sample: (i) the antimicrobial susceptibility profile of a pathogen in a mono-microbial sample; or (ii) the antimicrobial susceptibility profile of multiple types of pathogens in a poly-microbial sample; (iii) the antimicrobial susceptibility profile of a multiple-drug-resistant pathogen in a mono-microbial sample; or (iv) the antimicrobial susceptibility profile of each multiple-drug-resistant pathogens, in a poly-microbial sample. In yet a further embodiment, a defined set of concentrations for the panel of antimicrobials are used, and wherein the set of concentrations for the panel of antimicrobials provides for 13-1024 different concentrations of the antimicrobials from the panel of antimicrobials in order to carry out the antimicrobial susceptibility profile analysis of pathogens defined in (i), (ii), (iii) or (iv), and wherein each of the 13-1024 different concentrations contains only one type of antimicrobial from the panel of antimicrobials. In another embodiment, a defined set of concentrations for the panel of antimicrobials are used, and wherein the set of concentrations for the panel of antimicrobials provides for 13-1024 different concentrations of the antimicrobials from the panel of antimicrobials in order to carry out the antimicrobial susceptibility profile analysis of pathogens defined in (i), (ii), (iii) or (iv), and wherein each of the 13-1024 different concentrations contains one type or multiple types of antimicrobial(s) from the panel of antimicrobials. In yet another embodiment, the method further comprises steps (iv) and/or (v): (iv) measuring the growth of the unidentified and/or unknown pathogens within a pre-determined viability culture time after removing matrix interference components; and/or (v) measuring the growth of the unidentified and/or unknown pathogens within a pre-determined viability culture time after concentrating the pathogens in the unprocessed sample. In a further embodiment, the pre-determined viability culture time is selected from 5 min, 30 min, 1 hour, 2 hours, 3 hours, 6 hours, 12 hours, and 18 hours, or a time sufficient to generate about 100 CFU/ml. In another embodiment, the unidentified and/or unknown pathogens of step (iv) and/or (v) are concentrated in the unprocessed sample by centrifugating the unprocessed sample to pellet the unidentified and/or unknown pathogens and then removing supernatant. In yet another embodiment, for step (iv) and/or (v), the unidentified and/or unknown pathogens are indicated as being antimicrobial susceptibility profile growth of the unidentified and/or unknown pathogens is classified as no observed growth, limited growth, minimum growth, and relatively low growth. In a further embodiment, the antimicrobial susceptibility profile includes but is not limited to homogeneous microbial populations, heterogeneous microbial populations, pseudo-homogeneous microbial populations and pseudo-heterogeneous microbial populations. In another embodiment, the antimicrobial susceptibility profile includes but is not limited to the case where the majority of a heterogeneous microbial population is susceptible to a given antibiotic and a minority of the population is resistant, so the growth of the minority population will only be observed after the inhibited growth of the susceptible majority is observed. In a further embodiment, the method is fully automated by the use of a robotic handling system to carry out the method steps.

### BRIEF DESCRIPTION OF THE FIGURES

FIG. 1 presents representative ID results for target pathogens on the ID panel.

FIG. 2 presents representative AST results for target pathogens on the AST panel.

FIG. 3 provides total turnaround time of proposed direct-from-specimen assays (larger grey shaded box) compared to our current system (smaller grey shaded box) and other methods used in clinical microbiology.

FIG. 4A-C provides (A-B) calibration curves of configurable ID protocols with various TAT and LODs, and (C) AST responses to exposure times.

FIG. 5A-F presents (A-C) direct-from-specimen AST with 4-hr exposure for blood and 2-hr exposure for urine, and (D-F) AST responses to exposure times between resistant and susceptible strains.

FIG. 6A-C presents a (A) P. aeruginosa ATCC 27853 5×5 checkerboard with gentamicin (MIC: 1 μg/mL) and meropenem (MIC: 0.5 μg/mL). Assay results after 3.5 hours of incubation at 37° C. (B) A 3×3 checkerboard of gentamicin and meropenem on the same P. aeruginosa strain. (C) An exemplary embodiment of a 96-well checkerboard plate for 66 combinations. ABX are gray-scaled with different intensities to represent different concentrations.

FIG. 7 presents a polymicrobial ID/AST of CRE and MRSA.

FIG. 8A-F provides microbiological responses to short CST (2-hr) exposure for AST and long (16-24-hr) for time-kill.

FIG. 9A-B presents the components of an electrochemical sensor of the disclosure. (A) The 16-sensor array (2.5 by 7.5 cm) was microfabricated with a thin, optical-grade layer of gold electrodes deposited on plastic. Each sensor in the array contained three electrodes: a central working electrode, a circumferential reference electrode, and a short auxiliary electrode. (B) Detection strategy. 1 Bacterial lysis to release 16S rRNA target (black dashed line). 2 Hybridization of the target with the fluorescein (green circle)-labeled detector probe (blue line). 3 Hybridization of the target with the biotin (red circle)-labeled capture probe (orange line). 4 Binding of anti-fluorescein antibody conjugated with HRP to the target-probe sandwich. 5 Generation of current by transfer of electrons to the electron transfer mediator, TMB.

FIG. 10 presents a flow diagram illustrating an exemplary process to generate pathogen test samples for use in the various assays disclosed herein.

FIG. 11 presents a flow diagram illustrating an exemplary process to identify pathogen(s) from a pathogen test sample.

FIG. 12 presents a flow diagram illustrating an exemplary process to perform antimicrobial susceptibility testing of pathogens from a pathogen test sample.

FIG. 13 presents a flow diagram illustrating an exemplary process to assess multi-drug resistance of pathogens from a pathogen test sample.

### DETAILED DESCRIPTION

As used herein and in the appended claims, the singular forms “a,” “and,” and “the” include plural referents unless the context clearly dictates otherwise. Thus, for example, reference to “an antimicrobial” includes a plurality of such antimicrobials and reference to “the pathogen” includes reference to one or more pathogens or equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

All publications mentioned herein are incorporated herein by reference in their entirety for the purposes of describing and disclosing methodologies that might be used in connection with the description herein. Moreover, with respect to any term that is presented in the publications that is similar to, or identical with, a term that has been expressly defined in this disclosure, the definition of the term as expressly provided in this disclosure will control in all respects.

Unless defined otherwise, all technical and scientific terms used herein have the same meaning as commonly understood by one of ordinary skill in the art to which this disclosure belongs. Although many methods and reagents similar to or equivalent to those described herein can be used in the practice of the disclosed methods and compositions, the exemplary methods and materials are now described.

Children are more prone to injury than adults as they take more breaths per minute, and their breathing zone is closer to the ground. If they sustain burns or open wounds, they are more vulnerable to the effects of infectious agents and more susceptible to secondary infections. They also have thinner skin, which provides less protection and increases the chance of post-disaster skin and soft tissue infections. Natural disasters pose similar challenges to pediatric care; in the week following Hurricane Andrew, bacterial skin infections and open wounds were diagnosed more frequently in children, reflecting their increasing curiosity about their changing environment. Children require different antibiotics and different dosages to counter many chemical and biological agents. Currently, emergency care professionals have limited evidence-based guidelines or antimicrobial susceptibility testing (AST) information to assist them with the prescribing of medications for infants, children, and adolescents. Adverse drug events are common, particularly for antibiotics that are commonly prescribed to children in an emergency setting, such as ceftriaxone, clindamycin, and amoxicillin.

To date, PCR-based pathogen identification can be performed in less than 30 minutes, but no phenotypic AST exists that can be performed within a reasonable time frame (<30 min) directly from clinical samples in clinical microbiology laboratory settings. Researchers have demonstrated that AST results can be obtained using benchtop digital nucleic acid LAMP quantification of DNA replication to measure the phenotypic response of fast-growing Escherichia coli present within clinical urine samples exposed to an antibiotic for 15 min, but only highly resistant or susceptible strains were selected for testing. For slow-growing pathogens such as N. gonorrhoeae, which has a doubling time of about 60 min, a longer antibiotic-exposure incubation would be required. Additional researchers have demonstrated that quantifying changes in RNA signatures instead of DNA replication resulted in significant shifts (>4-fold change) in transcription levels within 5 min of antibiotic exposure. However, there was a wide range of control:treated ratio (C:T ratio) dispersion from highly susceptible strains with MICs at least seven 2-fold dilutions below the resistant break point. The C:T ratio can change from 2 to 6 with 8 strains with an MIC of 0.015 μg/mL and one strain with an MIC of 0.03 ug/mL, while the C:T ratio separation between the resistant and susceptible populations is only about 0.4. This indicates the limitation in clinical settings when not all susceptible strains have MICs that low.

Culture-based microdilution and disk diffusion tests are the gold standard for determining the susceptibility of causative bacteria to antibiotics. However, they are unacceptably slow (˜2 days) and laborious. There is always a valid concern about the detection sensitivity and matrix interference when developing a direct-from-specimen AST without the need of an overnight cultured isolate.

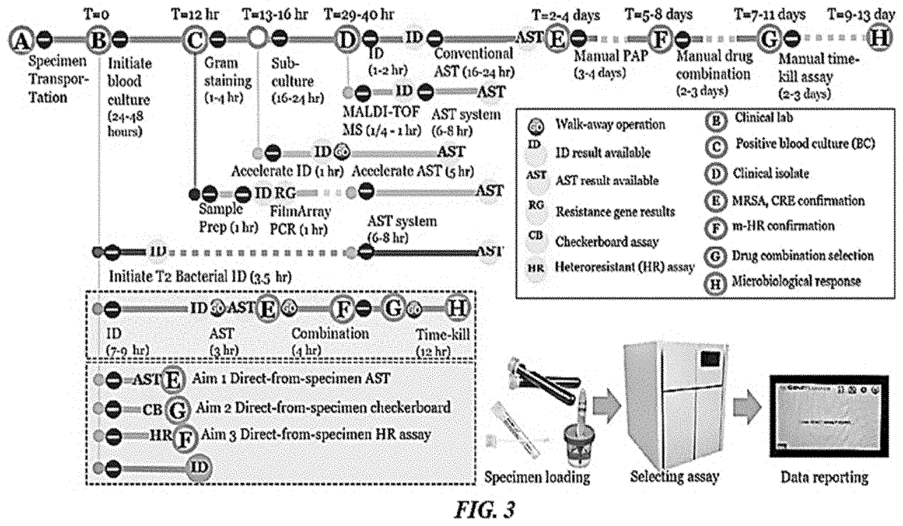

Molecular diagnostic assays are now available for direct testing of positive blood cultures (BCs), such as the T2 Bacteria® Panel, which is cleared by FDA and CE Marked in the EU. However, it does not provide either AST or resistance gene detection. A subculture for AST is still required, as indicated in the product label, for managing effective antimicrobial therapy. Current ID/AST reporting still takes more than a day. Based on a combination of biochemical reactions, microbial identification has been successively miniaturized and then automated with the bioMérieux VITEK2®, WalkAway MicroScan® (Siemens) or BD Phoenix® systems (FIG. 3 gray line). Being pivotal for the accurate management of patients with infectious diseases, identification has thus been accelerated significantly (FIG. 3 blue line). However, the selection of the right antibiotic treatment still requires time-consuming phenotypic AST according to FDA guidelines.

Despite a certain level of automation achieved over the last decades, obtaining AST results in modern clinical laboratories still requires more than 10 hours following bacterial isolation. As a result, one to three days of delay occur between the initiation of the empirical antimicrobial therapy and the AST result. Comparison with clinical trials using other approaches without the need of a clinical isolate such as Accelerate (FIG. 3 orange line) and FilmArray (FIG. 3 purple line) are listed in Table 1.

TABLE 1
Difference in AST category between ProMax system and disk diffusion reference method.

| Ciprofloxacin | Reference Results | | | Evaluation | |
|---|---|---|---|---|---|
| Test Results | S | I | R | Categorical Agreement | 132/143 = 92.3% |
| S | 72 | 2 | 1* | | |
| I | 1 | 0 | 3 | Reproducibility | 137/143 = 95.8% |
| R | 4^{h} | 0 | 60 | | |
| Test Results | S | I | R | Categorical Agreement | 148/155 = 95.5% |
| S | 89 | 0 | 3^{c} | | |
| I | 0 | 0 | 3 | Reproducibility | 149/155 = 96.1% |
| R | 1^{d} | 0 | 59 | | |
| Meropenem | Reference Results | | | Evaluation | |
| Test Results | S | I | R | Categorical Agreement | 148/155 = 95.5% |
| S | 89 | 0 | 3^{c} | | |
| I | 0 | 0 | 3 | Reproducibility | 149/155 = 96.1% |
| R | 1^{d} | 0 | 59 | | |

^{a}The algorithm cutoff for the susceptible category was too high, leading to a false susceptible result. Cutoffs have been adjusted since and further testing of this strain showed categorical agreement with the reference method.
^{b}(1)For some tests, the positive internal control on the sensor chip showed low signal, indicating poor quality, which affected the signal from the assay. Different batches of chips were tested thereafter, resulting in categorical agreement. (2) We also found that P. aeruginosa strains grow more slowly, leading to growth control fails or low signal overall. Starting concentration and incubation conditions have been adjusted accordingly, leading to consistent categorical agreements for this organism and other slow-growing organisms.
^{c}See Note 2b.
^{d}See Note 2a.
^{e}See Note 2a.

Currently, both FilmArray (FIG. 3 purple line) and T2 (FIG. 3 brown line) cannot report AST, so the total turnaround time (TAT) is determined by the availability of an additional AST system or conventional AST results (after purple and brown dash-lined). The completed and ongoing projects focus on streamlined ID/AST (FIG. 3 green box), so the AST will be performed only after a positive ID reporting from the set-aside pellet from the clinical specimens. Therefore, the susceptibility information will not be available until about 12 hours after the specimen loading onto the system (red stop sign).

While the concept of direct-from-specimen AST is appealing, there are significant challenges to this approach, and FDA has not seen enough scientific evidence to support this approach. The first challenge is that most growth-based susceptibility testing requires a standardized inoculum where a known concentration of organism is used for AST. In routine testing, the organism concentration is fixed, and it may be significantly higher than what is encountered in a clinical specimen which may be used for direct inoculation. An exception may be the urine culture, where patients with real infections commonly have more than 105 CFU/mL. Researchers have shown that urine can be used as an inoculum for rapid AST. This method employed a brief incubation period (˜120 min) followed by quantitative PCR designed to quantify growth. Pilot experiments showed that the assay was able to accurately determine Escherichia coli susceptibility to ciprofloxacin and trimethoprim within 3.5 h, however the susceptibility profiling algorithm was not correlated to the CLSI M100 categorical reporting. Methods disclosed herein overcome the forming challenges by doing susceptibility response dynamic trend/change at three different antimicrobials/pathogen ratios by inoculating the raw specimens in three dilutions as detailed above.

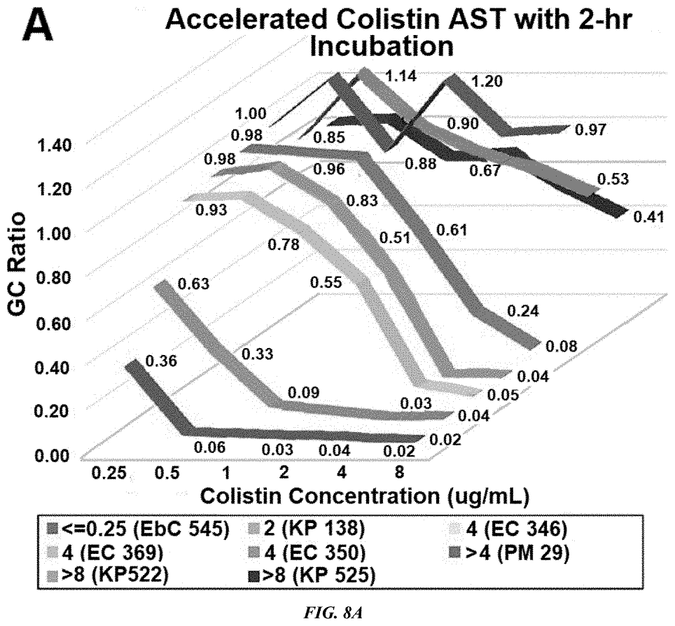

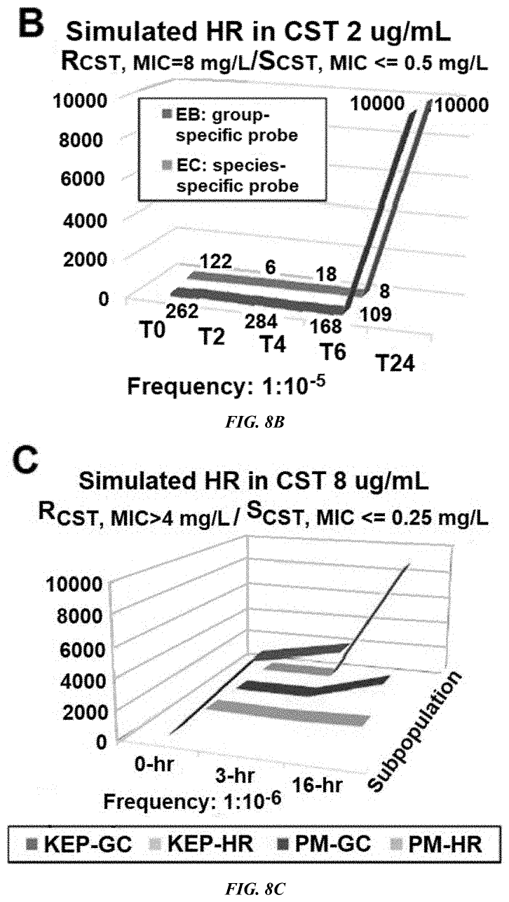

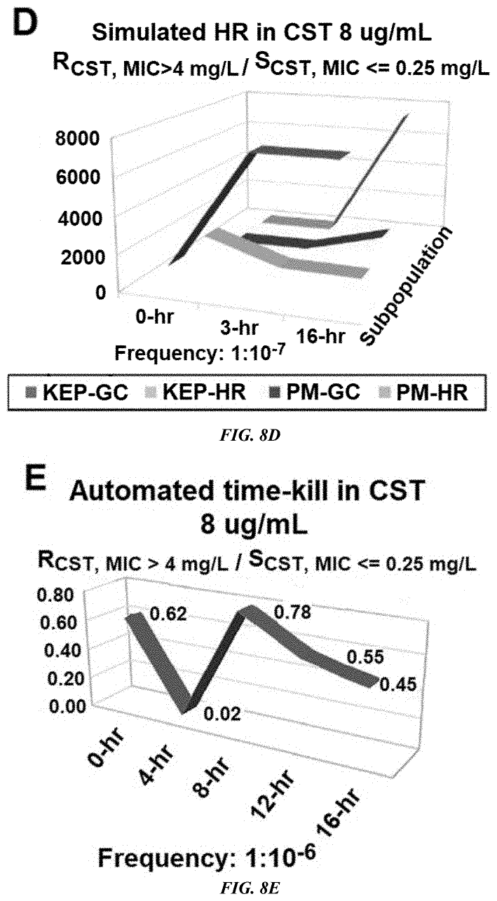

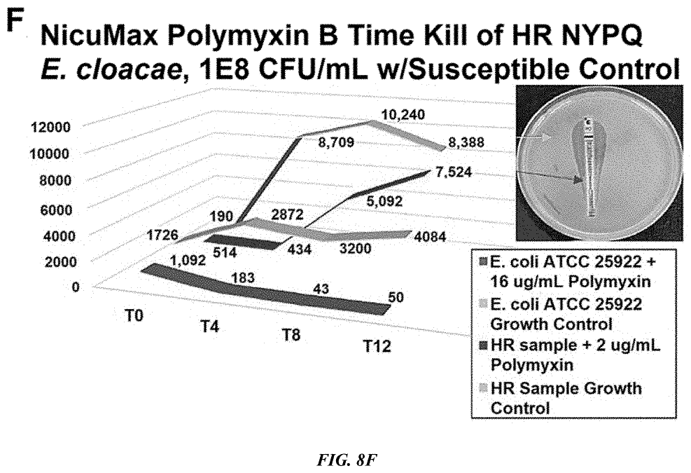

In direct contrast, the methods for assessing antimicrobial susceptibility profiling of unknown or unidentified pathogens disclosed herein can be performed directly from unprocessed specimens simultaneously on multiple samples and/or multiple specimen types on the same system by scanning the bar code on the sample collection tube. In FIG. 8A, a strong correlation of reduction of growth control (GC) ratio while increasing antibiotic concentrations indicates that the proposed direct-from-specimen susceptibility assays do not exhibit the same issue around the susceptible and resistant breakpoints. The methods of the disclosure provide for same-day direct-from-specimen pathogen identification (ID) and antimicrobial susceptibility testing (AST) to validate an evidence-based diagnosis of subjects in need of emergency assessment and antibiotic treatment. The methods of the disclosure can integrate robotic lab automation, rapid molecular analysis, genotypic pathogen quantification and phenotypic antibiotic susceptibility testing to dramatically improve the sensitivity and specificity of rapid, evidence-based pathogen identification (ID) and antimicrobial susceptibility testing (AST) directly from limited whole blood volume of infants in the nursery and NICU settings. The ID panel used in methods of the disclosure can provide identification of many types of pathogens, or for select pathogens, like Escherichia coli, Staphylococcus aureus (MSSA/MRSA), Staphylococcus epidermidis (CoNS), Enterobacter spp., Klebsiella spp., Serratia spp., and Pseudomonas spp.

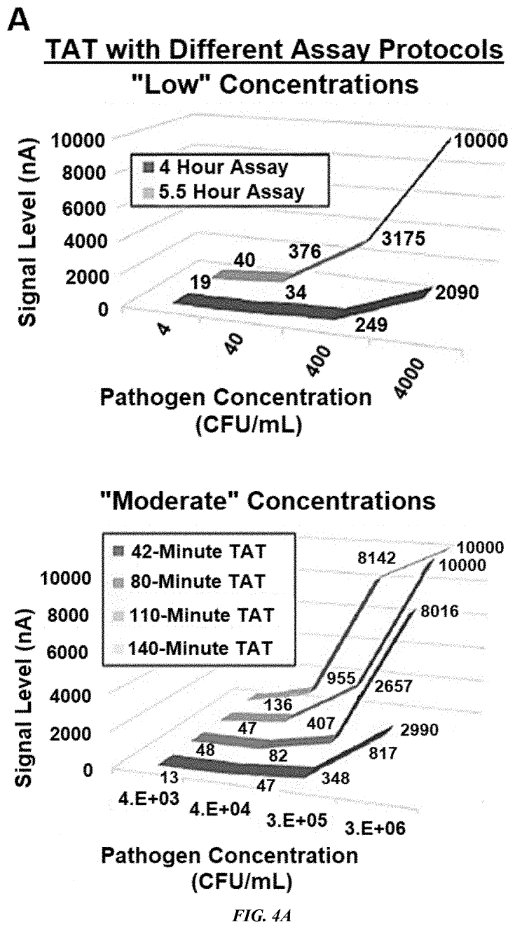

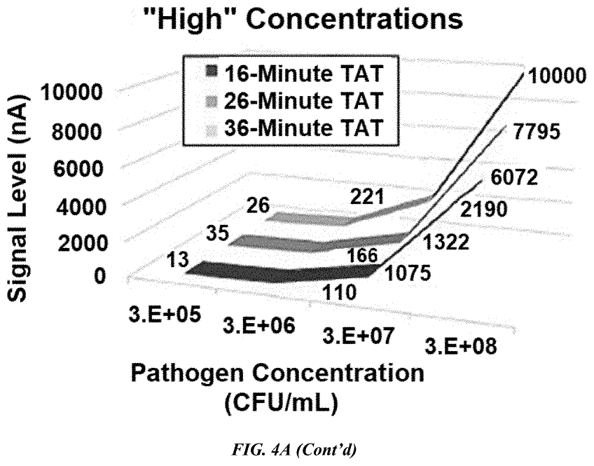

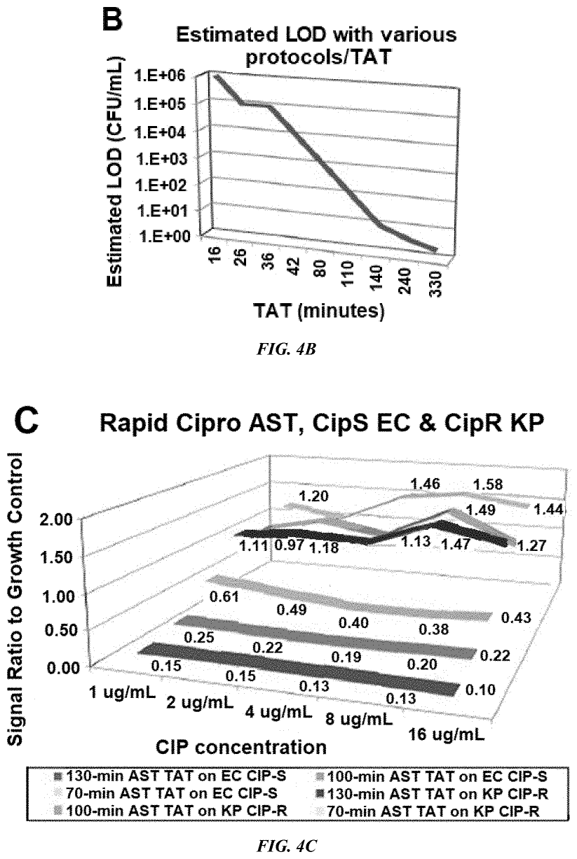

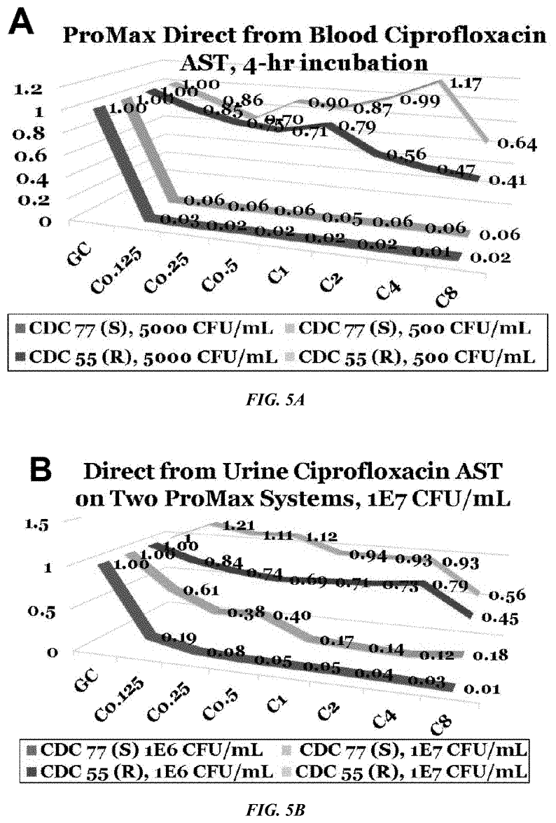

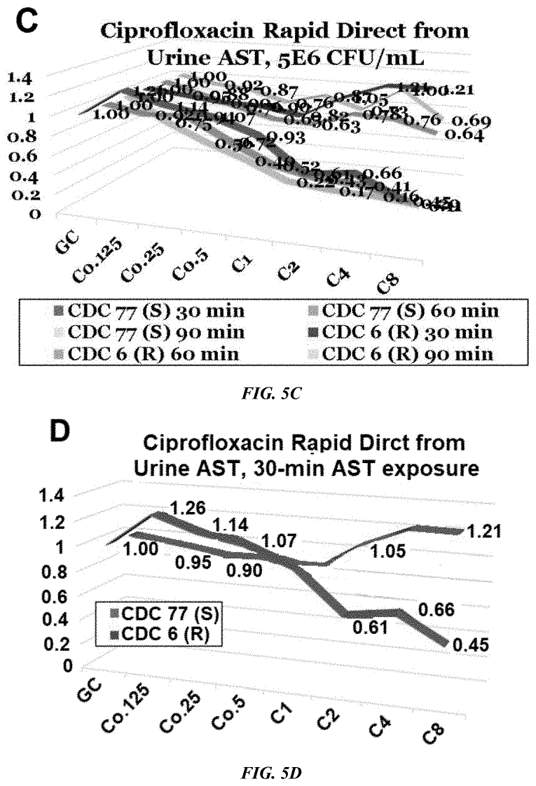

The direct-from-specimen AST disclosed herein will start with various types of specimens with an unknown concentration of pathogens, from 0 to >10⁸ CFU/mL. In order to determine the minimum assay time needed for quantification of RNA transcription at different levels of pathogen concentration, a correlation between the limit of detection (LOD) of the current molecular analysis platform (see FIG. 3 steps 1-17) with the assay turnaround time (TAT) was established (see FIG. 4). As shown in FIG. 4A, the TAT and dynamic range of ID can be configured to be from 16 minutes to 36 minutes by adjusting the analyte incubation time for higher target LODs as shown in FIG. 4B. Target pathogen enrichment and matrix component removal can be carried out by the built-in centrifugal module to achieve lower target LODs with TAT of 42 minutes to 110 minutes. For low-abundance pathogens and early infection diagnostics, additional viability culture steps with TAT of 240 minutes to 330 minutes can be included to achieve an LOD of <10 CFU/mL. The direct-from-specimen AST method disclosed herein will encompass these assay parameters. The BsiMax ID/AST system can adjust the antibiotic exposure time for direct-from specimen AST and adjust the matrix interference reduction procedures for specimens such as whole blood, urine, and urethral/vaginal swab. To assess the feasibility of direct-from-specimen AST, contrived urine and blood samples were used at three different concentration levels. Semi-automatic AST tests directly from whole blood and urine contrived samples were evaluated in FIG. 5 A-C. Additionally, the BsiMax ID/AST system can be programmed to conduct fully automated direct-from-specimen AST tests with multiple specimen types. With a 4-hour antibiotic exposure, resistant strains (CDC69) can be differentiated from susceptible strains (CDC77) (see FIG. 5A). Based on the trend of GC ratio changing along the increasing meropenem concentrations (1 to 64 μg/mL), the MIC would be reported as <1 μg/mL for CDC77 and 8 μg/mL for CDC 69, which agree with the MIC values from CDC AR Bank. To explore biological, chemical and molecular analytical limitations, shorter antibiotic exposure times were used to assess the separation of responses curves from both resistant and susceptible strains (see FIG. 5D-F).

Direct-from-specimen AST Analytical Validation Data Analysis. The current electrochemical-based biosensor measures the reduction current from cyclic enzymatic amplification of a horseradish peroxidase (HRP) enzyme label with TMB and H₂O₂. The resulting current signal can be estimated with the Cottrell equation. Each triple-response-curve signature are generated by overlaying three curves of all microbial/drug conditions with identified trends in GC ratios while increasing antibiotic concentrations, establishing a signature library corresponding to each inoculum concentration (see FIG. 5A-C). Changes in response signature are analyzed by the current algorithm with a trend of categorical classification change (such as always susceptible, from susceptible to resistant or always resistant), prior to the sample analysis. Contrived bacterial species are crosschecked by finding the closest matches in GC ratio trending of triple-response-curve signatures of each unknown bacterium to the ones in the signature library. The categorical classification of hundreds of different reference targets, pathogens without susceptibility profiles or even unknown pathogens can theoretically be identified and distinguished with the same triple-response-curve signature in this manner as demonstrated in FIG. 5. Categorical agreement is expected to be >95%.

Direct-from-specimen AST Lab Automation System Modification and Integration Approach. Automated AST instruments are extensively used in clinical microbiology labs in the US, replacing manual methods to perform gold standard microdilution or disk diffusion methods. These automated instruments still require the use of isolated bacteria grown in pure culture, and the susceptibility tests are based on measuring bacterial growth and turbidity changes. As a result, these automated technologies remain inherently slow and are severely limited by the low sensitivity of current detection methods. The lab automation system has been optimized to provide a streamlined workflow and quantitative results for direct-from-specimen AST, thus simplifying MIC determinations for pathogenic bacteria directly from clinical samples.

Direct-from-specimen AST clinical feasibility validation approach—The purpose of the direct-from-specimen AST clinical testing with remnant clinical specimens is to ensure and/or characterize the assay's AST accuracy to the causative pathogen AST reporting from a clinical microbiology laboratory. A BsiMax system is used to run the direct-from-specimen AST test on de-identified clinical remnant 150 urine, 150 swab and 150 whole blood samples received in the clinical microbiology lab. Categorical agreement (CA) occurs when the interpretation of the results of the reference method agree exactly with the interpretation of the BsiMax direct-from-specimen AST. Essential agreement is calculated according to the FDA Class II AST guidelines. Summary of the CA agreement of each antibiotic on the AST panel is presented in the following format: AST reporting format (min=minor discrepancies, maj=major discrepancies, vmj=very major discrepancies) Acceptance Criteria: (1) Overall ≥95.0% categorical agreement, (2) Major errors should be <3% of susceptible results, (3) Very major errors should be <2% of resistant results subject to sample size limitations.

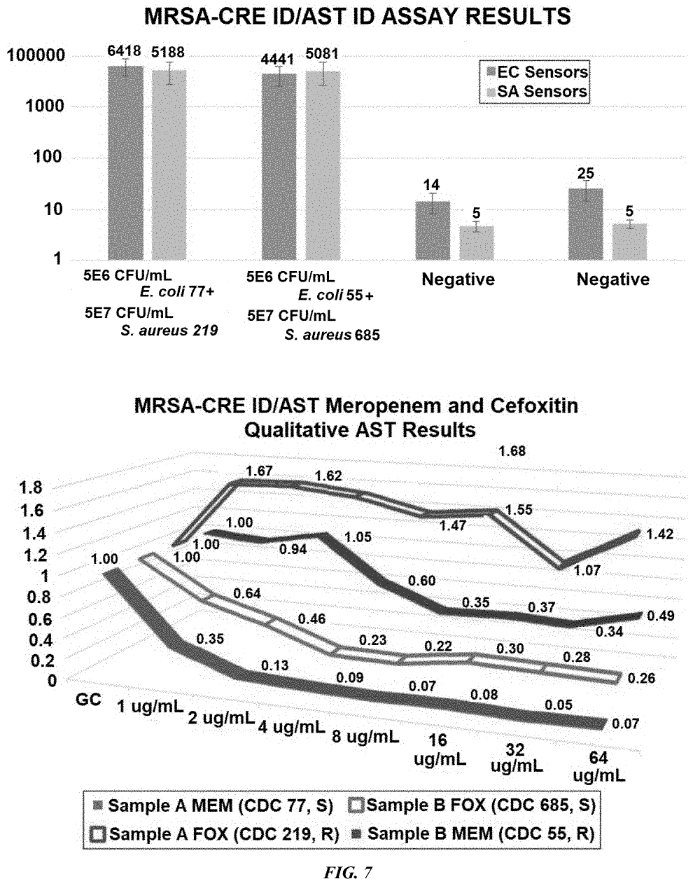

As demonstrated in FIG. 7, the tryptic soy broth (TSB) culture media supports the growth of both Gram-positive and Gram-negative bacteria for the proposed accelerated AST conditions. However, specimen-specific growth media may allow the maximum RNA transcription as seen in the current ID/AST protocols with MH media for urine and TSB for blood samples. If the categorical agreement is <90% for a particular specimen, a specimen specific culture media in the AST reagent kit is used.

Direct-from-specimen determination of combinational therapy for children with MDR infections Rationale. Six months into the COVID-19 pandemic, the first publication of drug cocktail therapy observed that patients with mild to moderate illness caused by the coronavirus between Feb. 10 and Mar. 20, 2020, appeared to improve more quickly if they were treated with a three-drug cocktail, compared with a group receiving just a two-drug combination. However, the drug combinations used in the COVID-19 clinical trial have been shown to reduce the mortality and need for intensive respiratory support of patients with SARS in a clinical trial in 2003. During an emergency or an unprecedented pandemic, such proven or previously validated drug combinations might not be available for reference. In patients with presumed acute infections, initial empirical antibiotic therapy, before the results of pathogen identification and susceptibility testing are available, is selected based on individual patient characteristics, clinical differential diagnosis, place of infection (i.e., community versus hospital-acquired) and non-patient-related epidemiological data such as local bacterial susceptibility rates (SRs). The choice of empirical antibacterial therapy in hospitalized patients is guided by institution-specific cumulative antibiogram reports, which compile mean SRs of bacterial isolates collected from mostly adult patients previously treated at the same institution. Hospital-wide cumulative antibiograms from an adult-majority population may not sufficiently support informed decision-making for optimal treatment of pediatric patients in an emergency, especially for an emerging pandemic with MDR or unknown pathogens.

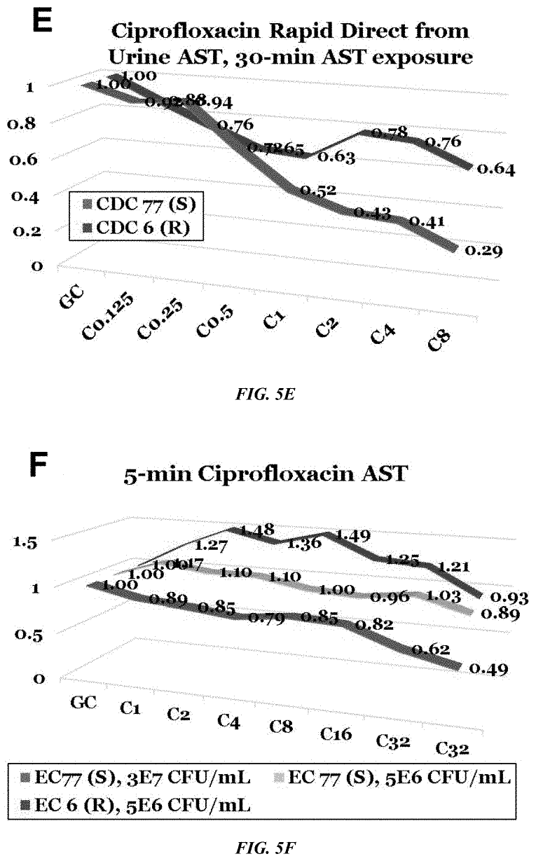

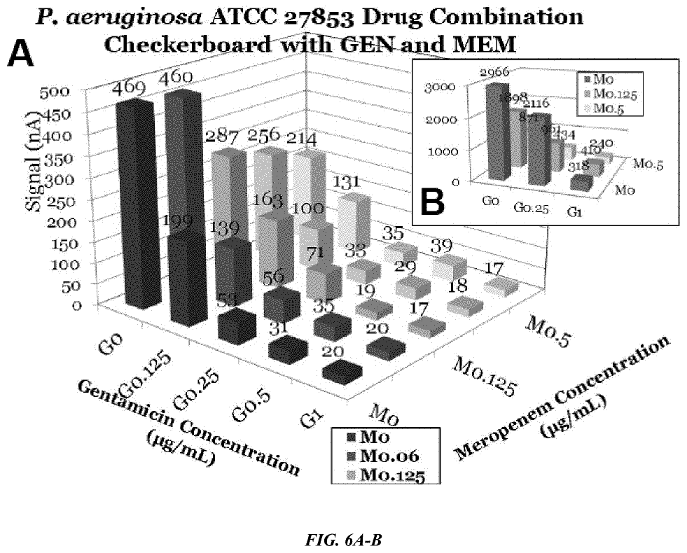

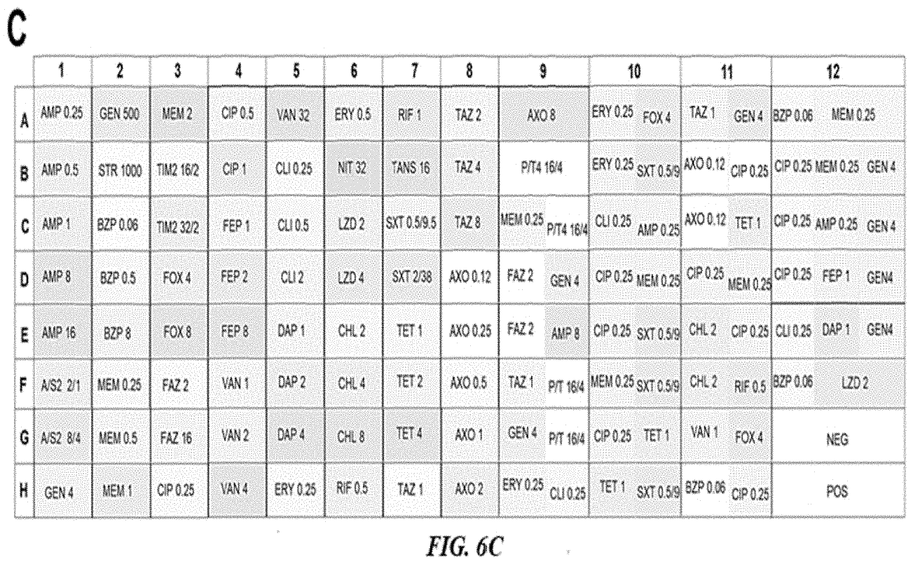

The disclosure provides for the establishment and validation of an automated protocol to assess combinations of two or more antimicrobials to assess antimicrobial susceptibility directly from pediatric patients' specimens during an emergency or unprecedented pandemic. The direct-from-specimen AST protocols discussed above are expanded into a 96-well plate-based checkerboard test to assess 66 preselected combinations of 25 antibiotics as illustrated in FIG. 6C. Preliminary results of checkerboard tests (5×5 and 3×3 of MEM and GEN) using the accelerated molecular quantification of RNA transcription of microbiological response are shown in FIG. 6 A-C to 25 and 9 antibiotic combination exposures were demonstrated in FIG. 6A-B. To improve the potential utility, flexibility and adaptability, the following modifications and developments are implemented to the BsiMax lab automation system.

Polymicrobial infections (CRE and MRSA). Polymicrobial infections are particularly common in critically ill, high-risk, and bed-bound patients in healthcare settings. Because of the potential risk and high treatment cost, it is imperative that the system can reliably detect polymicrobial infections and correctly assess the phenotypic AST results to ensure that the presence of multiple pathogens in a single patient sample will not negatively affect the growth during the incubation portion of the AST testing or the results themselves. Furthermore, in the case of polymicrobial AST, the system uses species-specific sensor chips to accurately distinguish between the two present infecting species in order to provide accurate treatment diagnosis (see FIG. 7) using the ID/AST protocol in the green box in FIG. 3. The occurrence of multidrug-resistant infection is a major concern in hospital-acquired infections (HAIs), especially in high-risk populations. In addition to MRSA, other resistant strains have been associated with HAIs that are often not responsive to even a broad range of antibiotics. In FIG. 7, contrived tryptic soy broth samples, two negative and two seeded polymicrobial samples, were tested to verify the feasibility of a streamlined ID/AST for BsiMax®. Specifically, the target of these samples was either a carbapenem-resistant Enterobacteriaceae (CRE) or methicillin-resistant S. aureus (MRSA) strain. Each sample is tested with the ID portion of the assay and the samples that test positive proceed to the AST portion of the assay, which tests each sample for meropenem and cefoxitin as an indicator of carbapenem and methicillin resistance respectively. The identities of the bacterial strains tested are included in the top bar chart. The direct-from-specimen antimicrobial response of polymicrobial contrived or clinical remnant samples are assessed with the checkerboard 96-well plate to be validated in MRSA and CRE as shown in FIG. 7 with species-specific sensor chips. Direct-from-specimen determination of combinational therapy for subjects with MDR analytical and clinical feasibility validation. The clinical remnant specimen collection workflow, as well as, the inclusion/exclusion criteria are the same as detailed herein.

The direct-from-specimen AST is used for monoclonal infections resistant to first line antibiotics, and the direct-from-specimen checkerboard assay establishes effective combinational therapy for difficult-to-treat polymicrobial and/or multidrug resistant (MDR) infections. The direct-from-specimen early detection of HR focuses on a less frequent event where the outcome from the AST and MDR tests would fail due to HR, which is defined as a subpopulation of the bacterial population demonstrating phenotypic resistance without any genotypic changes compared with the parent population. Undetected and untreated HR of causative isogenic bacteria from individual patients is a major cause of unexplained antibiotic treatment failure and development of antimicrobial resistance (AMR) in CRE infections. Colistin is the last resort therapy option for bacterial infections and therapeutic dosing of antibiotics without considering the highly resistant subpopulations of an HR isolate will cause CRE treatment failure and select for the more resistant subpopulations. Resistance to last-line drugs such as colistin continues to emerge in clinical CRE infections. Although colistin resistance is rare, colistin treatment failures are not uncommon, raising concern that colistin heteroresistance may be a basis for treatment failure.

In some HR bacterial strains, a resistant sub-population that comprises as low as 10⁻⁸ of the entire bacterial population can be measured from the exposure of a homogeneous isogenic culture of a susceptible bacterial population to sub-lethal dosages of antibiotics to avert selective pressure of AMR. Lab studies have identified HR with population analysis profiling (PAP) from all clinical isolates from patients with the last dose of colistin exposure within 0-18 days, and it is possible that the resistant proportion of the HR bacterial population might be selected and become predominant during colistin therapy, leading to treatment failure. Microbiologists at Emory Health Sciences (Atlanta, Ga., USA) found that within an isolate, the subpopulations resistant to different antibiotics were distinct, and over 88% of CRE isolates exhibited some degree of HR to multiple antibiotics (m-HR). However, no HR studies correlate the rate of heteroresistance to the rate of very major errors (VME) as defined by FDA, CLSI or EUCAST guidelines.

The disclosure further provides for the use of robotic systems to carry out the methods of the disclosure such as the UtiMax or BsiMax/UtiMax or BsiMax from GeneFluidics, Inc. of Irwindale, Ca., USA. The robotic systems allow for generating AST and other results without the need of a separated culture system, a concentration system, a purification system or having to perform repeated assays. As a result, it is possible to quickly and accurately identify if a pathogen is present, the identity of pathogen(s) that are present, and the susceptibility of pathogen(s) to pre-selected antimicrobials with practical levels of expense, assay time and manpower.

This ability to quickly characterize the pathogens can revolutionize treatment with antimicrobials such as antibiotics, Cefepime, and Meropenem. For instance, knowing quickly the AST of the pathogens(s) can prevent the prescription of antimicrobials in situations when they are not effective. Additionally, knowing the identity of pathogen(s) that are present allows for the prescription of antimicrobials that are known to be effective against the pathogen(s). Further, the quick availability of these results permits these prescriptions to be made in a time period that is most efficacious for the patient. For instance, these prescriptions can often be made in several hours. As a result, these prescriptions can generally be made on the same day a sample is taken from the patient, while the patient is on the way home from the site where the sample was taken, or while the patient is still at the site where the sample was taken. Since antimicrobials are tailored based upon the AST of the pathogens in the subject's sample, the most effective antimicrobial therapies can be administered, including for MDR pathogens and HR pathogens.

The ability to quickly know the AST of the pathogen(s) present in a sample can further permit screening of the most effective antimicrobials for the particular pathogen(s) before the prescription of an antimicrobial is made. For instance, the sample taken from a patient can be cultured in the presence of the antimicrobial(s) that are selected as being effective against the identified pathogen(s). In some instances, these susceptibility cultures are performed using different concentrations of the identified antimicrobial(s). The same assay technologies can be used to measure growth of the pathogen in these susceptibility cultures. These results can be compared to identify the antimicrobial's susceptibility, and, in some instances, Minimum Inhibitory antimicrobial Concentrations (MICs) of the identified pathogens. The antimicrobial to which the pathogen is most susceptible can then be prescribed. Additionally or alternately, the dosage of the prescription can be based on the Minimum Inhibitory antimicrobial Concentration (MICs). For instance, when lower concentrations of an antimicrobial are shown to be effective against a pathogen, lower doses of the antimicrobial can be prescribed. When higher concentrations of an antimicrobial are shown to be effective against a pathogen, the prescribed dosage of the antimicrobial can be increased. As a result, the initial prescription given to a patient can be experimentally shown to be effective against the strain of pathogen identified in raw sample taken from a patient. The use of experimental results to tailor the prescription to the identified pathogen, the proliferation of antimicrobial-resistant pathogens can be further reduced. Examples of pathogens or pathogens that can be characterized using the methods of disclosure include, but are not limited to, Actinomyces israelii, Bacillus anthracis, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, Yersinia pseudotuberculosis, Absidia corymbifera, Absidia ramose, Achorion gallinae, Actinomadura spp., Ajellomyces dermatididis, Aleurisma brasiliensis, Allersheria boydii, Arthroderma spp., Aspergillus flavus, Aspergillus fumigatu, Basidiobolus spp, Blastomyces spp, Cadophora spp, Candida albicans, Cercospora apii, Chrysosporium spp, Cladosporium spp, Cladothrix asteroids, Coccidioides immitis, Cryptococcus albidus, Cryptococcus gattii, Cryptococcus laurentii, Cryptococcus neoformans, Cunninghamella elegans, Dematium wernecke, Discomyces israelii, Emmonsia spp, Emmonsiella capsulate, Endomyces geotrichum, Entomophthora coronate, Epidermophyton floccosum, Filobasidiella neoformans, Fonsecaea spp., Geotrichum candidum, Glenospora khartoumensis, Gymnoascus gypseus, Haplosporangium parvum, Histoplasma, Histoplasma capsulatum, Hormiscium dermatididis, Hormodendrum spp., Keratinomyces spp, Langeronia soudanense, Leptosphaeria senegalensis, Lichtheimia corymbifera, Lobmyces loboi, Loboa loboi, Lobomycosis, Madurella spp., Malassezia furfur, Micrococcus pelletieri, Microsporum spp, Monilia spp., Mucor spp., Mycobacterium tuberculosis, Nannizzia spp., Neotestudina rosatii, Nocardia spp., Oidium albicans, Oospora lactis, Paracoccidioides brasiliensis, Petriellidium boydii, Phialophora spp., Piedraia hortae, Pityrosporum furfur, Pneumocystis jirovecii (or Pneumocystis carinii), Pullularia gougerotii, Pyrenochaeta romeroi, Rhinosporidium seeberi, Sabouraudites (Microsporum), Sartorya fumigate, Sepedonium, Sporotrichum spp., Stachybotrys, Stachybotrys chartarum, Streptomyce spp., Tinea spp., Torula spp, Trichophyton spp, Trichosporon spp, and Zopfia rosatii.

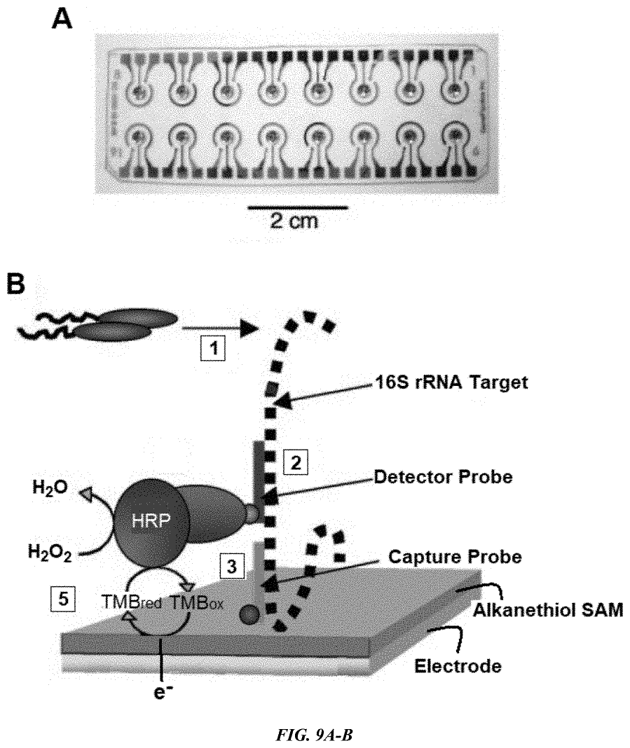

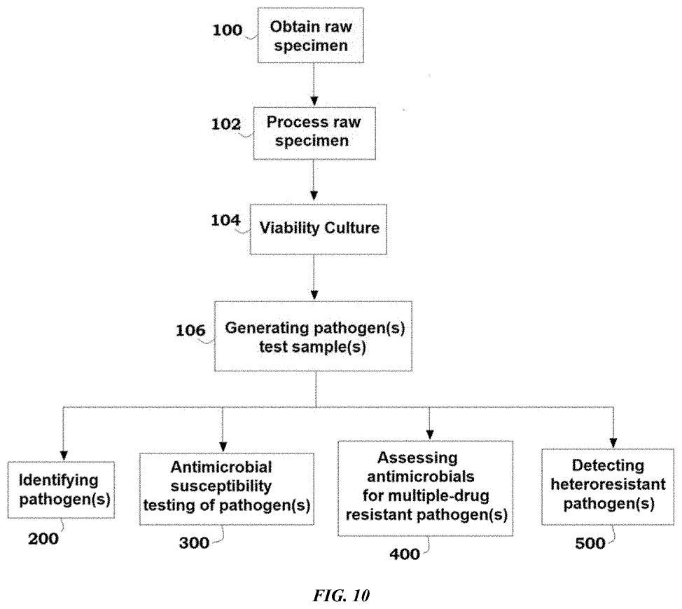

The assays described herein can be used to characterize unknown or unidentified pathogens directly from raw specimens, without having to generate clinical isolates. The assays provide for the identification of the pathogens, determining AST, determining combinational therapy for MDR infections, and for the early detection of heteroresistant pathogens. The disclosure provides a method of preparing raw samples to generate concentrated pathogen samples that can be used in the various assays disclosed herein. FIG. 10 is a flow diagram illustrating this method to generate pathogen samples for use in the various assays disclosed herein. The method can be fully or partially performed by a robotic system such as the UtiMax or BsiMax/UtiMax or BsiMax from GeneFluidics, Inc. of Irwindale, Ca., USA. When such a system is used, a user receives a raw sample. At process block 100, the user can load the raw sample into the system. The raw sample can be taken from a raw source of pathogens such as food, the environment, or a biological source. Alternately, the raw sample can be taken from a patient such as a person, animal that is living or dead. Examples of raw samples taken from a patient include, but are not limited to, whole blood, urine, nasal swab, saliva, and Cerebrospinal fluid (CSF). The raw sample can be in a container such as a sample tube. The system can read an indicator on the container such as a label, bar-code, or the like on sample tube to identify one or more features about the raw sample. For instance, the system can read a bar-code on the container. The bar-code can identify the type of raw sample to the system. When the raw sample is taken from a source other than a patient, the type of raw source can be identified by the identifier on the container or entered by a user using a graphic user interface (GUI) on the system. The system can identify the one or more assays that are associated with the identified raw sample. The system can execute the remainder of the FIG. 9 flow diagram so as to perform the one or more assays directly from the raw sample, including identifying the pathogen(s) in the raw sample 200, determine AST of the pathogen(s) 300, performing checkboard assay for MDR pathogens 400, and detection of heteroresistant pathogens 500.

When a robotic system, such as Proteus, prepares the assay sample, the system can automatically perform the entire assay from receiving the raw sample, through preparing the assay sample to testing of the assay sample. When the assay sample is prepared and the assay is performed, the UtiMax or BsiMax robotic system accesses vials containing reagents for preparing the assay sample from the raw sample from the UtiMax or BsiMax reagent kits located in designated reagent rack(s). For instance, the reagents can include lysing solutions, buffers, enzyme, AST medium, and TMB. The UtiMax or BsiMax robotic system includes a centrifuge for performing concentration operations on one or more samples prepared by the UtiMax or BsiMax robotic system. The UtiMax or BsiMax robotic system can transfer lysates and the needed reagents onto sensor arrays comprising a plurality of electrodes. The UtiMax or BsiMax robotic system can incubate the test sample on the sensor followed by one or more optional operations such as washing, drying, and enzyme incubation. The UtiMax or BsiMax robotic system can perform electrochemical reading by adding TMB substrate onto each sensor and operating a multi-channel potentistat connected to the sensors. For instance, the UtiMax or BsiMax robotic system performs amperometry or cyclic voltammetry on the sensors. The UtiMax or BsiMax robotic system provides the results to a user. For instance, the UtiMax or BsiMax robotic system can indicate to a user the pathogen species identified by the system based on the sensor chip used for testing, the AST of the pathogens, etc. It can also indicate if multiple pathogens have been detected, and whether these pathogens have multi drug resistance or detect at an early timepoint heteroresistant pathogens. and, can often identify each of the species detected. Additionally, if the pathogen species is not identified by one of the species-specific sensors, it can indicate whether the tested sample contains a Gram-negative or Gram-positive sample. The UtiMax or BsiMax will also indicate whether the universal sensor that will detect any bacteria has produced a signal.

When using a robotic system such as the UtiMax or BsiMax to perform an assay, the testing time period from a user receiving the raw sample to a user receiving the results of the assay (an indication or positive or negative and/or concentration of pathogen) is generally on the order of 30-360 minutes. When manually performing an assay, the testing time period from a user receiving the raw sample to a user receiving the results of the assay (an indication or positive or negative and/or concentration of pathogen) is generally on the order of 30-360 minutes. Accordingly, the assays described above can be performed in a testing time period greater than 30 minutes, 60 minutes, or 120 minutes and/or less than 4 hours, 5 hours, or 6 hours.

The preparation of the target pathogen sample from the raw sample and the subsequent testing can be performed on site. For instance, since the testing can be performed using robotic systems such as UtiMax or BsiMax, the testing can occur in the same room, building, or medical complex where the raw sample was taken and/or where the assay sample was prepared from the raw sample. As a result, the time delay associated with transportation of the raw sample to an off-site location can be removed. Further, the total time to generate results from the pathogen identification phase are the cumulative times of the time to prepare the viability sample from the raw sample, the viability culture time, the time to prepare the assay sample from the aliquoted portion of the viability sample, and the time to test the assay sample. Once a raw sample becomes available, the viability sample can generally be generated in times less than 360 minutes, 180 minutes, or 30 minutes and/or greater than 0 minute, 30 minutes, or 180 minutes inside UtiMax or BsiMax. The assay sample can generally be prepared from an aliquoted portion of a viability sample in times less than 5 minutes, 2 minutes, or 1 minute and/or greater than 5 seconds, 15 seconds, or 1 minute. Accordingly, the time period between taking the raw sample and completing the pathogen characterization can be less than 120 minutes, 270 minutes, or 450 minutes after taking the raw sample. Accordingly, it becomes possible to receive the results of the assays in a time period less than 30 minutes, 180 minutes, or 360 minutes after taking the raw sample.

The AST for the pathogen(s) is performed before an antimicrobial is prescribed and the results of the antimicrobial susceptibility testing are taken into account when prescribing the antimicrobial. In an antimicrobial susceptibility testing phase, the susceptibility of the identified pathogen(s) to an identified antimicrobial is tested. For instance, an antibiogram can be established at each hospital. The antibiogram can be taken into account when making the prescription. For instance, a physician, pharmacist, user, technician, or other person authorized to write prescriptions can prescribe the antimicrobial to which the pathogen(s) is most susceptible or can refrain from prescribing any antimicrobials when the pathogen(s) are not susceptible to any of the identified antimicrobials.

At process block 102, a raw specimen is processed to remove matrix interfering components. Optionally, a purifying operation can be performed to reduce the number and/or concentration of at least one type of cells other than the one or more pathogens that are the target of the identified assays (target pathogens). For instance, when the raw specimen includes host cells in addition to the target pathogens, the purifying operation can be performed so as to reduce the concentration and/or number of the host cells. For instance, all or a portion of the raw sample can be lysed so to reduce the number and/or concentration of non-target pathogens in the sample that includes or consists of all or a portion of the raw sample. As an example, raw samples of whole blood include red blood cells (RBCs) that are not target pathogens. Accordingly, an RBC lysing agent can be used on the raw sample to selectively lyse RBC, but not target pathogens. Suitable RBC lying reagents include, are but not limited to, 1×RBC Lysis Buffer (ThermoFisher cat. no. 00-4333), ACK Lysing Buffer (ThermoFisher cat. no. A1049201), and Red Blood Cell Lysis Buffer (Roche 11814389001). Alternatively or additionally, an affinity pull-down, magnetic or Sepharose beads covered in antibodies or binding molecules that specifically bind surface antigens of target pathogens are added to the raw specimen, and by use of magnets or centrifugation the beads are isolated and the target pathogens are liberated.

At process block 104, the target pathogens are cultured in culture medium so as to increase the concentration of viable target pathogens in the sample. The viability culture time can be from 35 minutes to 330 minutes. In some instances, the viability culture time is a function of a desired LOD level or a clinical cutoff. Generally, process block 102 and process block 104 has an assay turnaround time (TAT) from 42 minutes to 110 minutes. For low abundance pathogens and early infection diagnostics, TAT for process block 102 and process block 104 can be from 240 minutes to 330 minutes in order to achieve an LOD of <10 CFU/mL.

In the above disclosure, the viability culture time is the minimum period of time over which the viable pathogens are cultured before preparing the pathogen test sample. For instance, a viability culture time of 3 hours indicates that viable pathogens have been cultured for at least 3 hours before the aliquoted portion of the viability sample is taken.

Using the above method of carrying out the viability culture has provided very low Limits of Detection (LOD) even when using very low culture times. For instance, for raw samples of whole blood, a LOD of less than 100, or 10 CF/mL have been achieved when using culture times less than 2 hours, or 3 hours. For raw samples of urine, a LOD less than 10,000 CFU/mL have been achieved when using culture times less than 1.5 hours and LOD less than 3E4 CFU/mL have been achieved when using culture times less than 30 minutes. For raw samples of culture media, LOD less than 10,000 CFU/mL have been achieved when using culture times less than 30 minutes and LOD less than 50,000 CFU/mL have been achieved without any viability culture.

The studies presented herein indicate that the pathogen concentration in raw samples is typically at the lower end of the possible concentration range. For instance, pathogen concentration in raw samples is typically on the order of <10 CFU/mL for blood, >10,000 CFU/mL for urine, <2 CFU/mL for lake water, <100,000 CFU/gram for cooked meat. The viability culture time can be selected to provide a LOD that is less than these concentration levels to ensure that any of the target pathogen in the raw sample is detected. Additionally, the viability culture time can be a function of the raw sample. For instance, for raw samples of whole blood, the viability culture time can be less than 5 hours, 4 hours, or 3 hours and greater than 0 hour, 1 hours, or 2 hours. For raw samples of urine, the viability culture time can be less than 4 hours, 3 hours, or 2 hours and greater than 0 hour, 30 minutes, or 60 minutes. For raw samples of culture media, the viability culture time can be less than 4 hours, 3 hours, or 2 hours and greater than 0 hour, 30 minutes, or one hour.

The above viability culture times can reduce the total assay time. A total assay time can be the period of time that lapses between receiving the raw sample and characterizing the target pathogen in the various assays described herein. When a robotic system is employed to do the viability culture and the corresponding assay(s), the total assay time can be the period of time that lapses after loading the raw sample into the system. Suitable total assay times include, but are not limited to, times less than 6 hours, 2 hours, or 30 minutes. As noted above, in some instances, the raw sample serves as the test sample and culturing is not required. In these instances, the total assay time can be substantially reduced. For instance, the total assay time can be less than 30 minutes, one hour, or two hours.

At process block 106, enriched viable target pathogen(s) can be concentrated by a variety of techniques including, but not limited to, filtering, sedimentation and/or centrifugal separation techniques such as spinning, centrifuging, pipetting, supernatant removal, and cellular pelleting. Centrifuging is an effective approach for concentrating target pathogens such as bacteria because bacteria are heavier and bigger than hemoglobin, lipids, and ions. Accordingly, centrifuging of the enriched sample causes pathogens such as bacteria to concentrate in the centrifuge pellet rather than the supernatant. As a result, the supernatant can be separated from the centrifuge pellet and the centrifuge pellet can serve as source of pathogen test samples for carrying out the various pathogen characterization techniques, including identifying target pathogen(s) in the raw specimen 200, determining AST of the target pathogen(s) 300, performing a checkboard assay for MDR pathogens 400, and detecting heteroresistant pathogens 500. For each of the foregoing assays, a portion of the pathogen test sample can be used to carry out each of the assays. Alternatively, if only one of the foregoing assays, e.g., identification of the target pathogens, is going to be carried out then the entire test sample can be utilized.

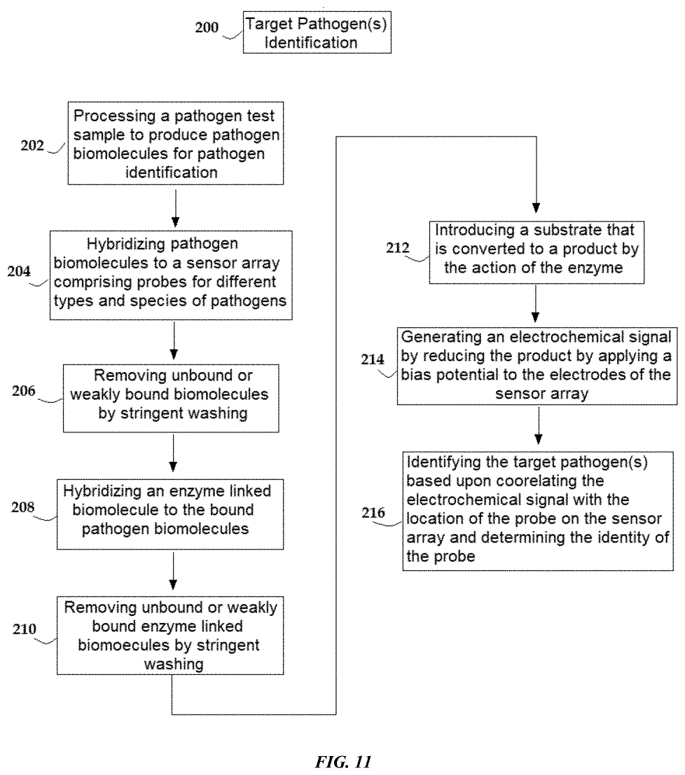

As shown in FIG. 11, the disclosure also provides methods to identify pathogens from a pathogen test sample 200. Typically, the pathogens are unknown or not identified in the raw specimen. However, if there is a primary indication or preliminary prognosis that certain target pathogens are present in the sample, then the methods of disclosure can be used to confirm or validate that indication or prognosis.

At process block 202, the pathogen test sample is treated, typically with lysis buffer, sonication, Dounce homogenization, or the like to disrupt the pathogen cell wall and/or membranes to release biomolecules, including but not limited to, nucleic acids (e.g., RNA, DNA), proteins, lipids, carbohydrates, or combinations of any of the foregoing. A typical protocol can include addition of cell lysis buffer, and other reagents, such as inhibitors to proteases, RNases and/or DNases; buffers; stabilization agents; etc. In a particular embodiment, the pathogen test sample is processed to isolate or recover target pathogen RNA (e.g., 16S rRNA).

At process block 204, the pathogen biomolecules are bound to a sensory array comprising probes for different types and species of pathogens. Typically, each electrode of the sensor array is bound or anchored to a probe that has a different pathogen biomolecule specificity than the other probes. The probes can be any number of typical capture ligands, including oligo- or polynucleotides with specifically defined sequences (e.g., sequences for species specific 16S rRNA); antibodies; receptor ligands; receptors; aptamers, nucleotide binding proteins; etc. The surface of the electrode that is bound to the probe typically comprises a highly conductive material, like gold. The electrodes of the sensor array are typically spaced from each other so as to avoid cross contamination. The pathogen biomolecules can be manually dispensed to the electrodes of the sensory array, or can be dispensed using automated liquid handlers, such as liquid handlers of the UtiMax or BsiMax robotic systems. The pathogen biomolecules are then bound to the probes under conditions that promote binding of the probe to the pathogen biomolecules, or vice versa. To minimize nonspecific binding of pathogen biomolecules to the probes then increasingly stringent conditions can be used. For example, if the probes are oligonucleotides or polynucleotides and the pathogen biomolecules are RNA then stringent hybridization conditions can be used. In a particular embodiment, the probes bound to the electrodes of the sensor array are oligonucleotides or polynucleotides comprise sequences that bind to 16S rRNA in a species-specific or group-specific manner.

At process block 206, unbound or weakly bound pathogen biomolecules are removed from the electrodes by using a washing process. The stringency of the washing conditions can be used to remove all but the most strongly bound pathogen biomolecules (i.e., the most complementary to the probe). The reagents and wash conditions are dependent on the pathogen biomolecules being captures. For example, heat can be used with polynucleotides, while detergents can be used with proteins. The number of washes performed can be empirically determined, but typically range from 1× to 10×. If the pathogen biomolecules are bound to the probes under stringent or very stringent conditions, minimal to no washes may be utilized. Alternatively, if the pathogen biomolecules are bound to the probes under non-stringent conditions more washes may be needed. Optional drying steps may be used prior to addition of an enzyme at process block 208.

At process block 208, an enzyme in solution is then dispensed to each electrode of the sensor array. The enzyme used is typically an enzyme which can oxidize a substrate to make a product. Examples of such enzymes, include but are not limited to, horse radish peroxidase (HRP), glucose oxidase, xanthine oxidase, galactose oxidase, and lactate oxidase. The type and nature of the substrate is dependent on the substrate suitability for the enzyme used in the process. The enzyme disclosed herein is typical conjugated to a second probe that can bind to target pathogen biomolecules. Accordingly, pathogen biomolecules bound to probes attached to the electrodes are further bound by the second probe (i.e., the pathogen biomolecules are sandwiched between the two probes). The second probe can be any number of typical ligands, including oligo- or polynucleotides with specifically defined sequences (e.g., universal sequences for 16S rRNA); antibodies; receptor ligands; receptors; aptamers; nucleotide binding proteins; etc. Choice of the second probe is largely dependent on the identity of the target biomolecule bound to the probes of the electrodes. For example, the second probe can be an antibody if the target biomolecule is a protein; or, alternatively, be a nucleotide sequence that hybridizes to complementary sequences of pathogen nucleotides (e.g., pathogen RNA). In a particular embodiment, the second probe is an oligonucleotide or polynucleotide that binds to a universal 16S rRNA sequence (i.e., a sequence that is found in most if not all pathogens). In a further embodiment, the second probe is conjugated to HRP.

At process block 210, unbound or weakly bound second probes bound to pathogen biomolecules are removed using a washing process. The stringency of the washing conditions can be used to remove all but the most strongly bound second probes to pathogen biomolecules (i.e., the most complementary to the probe). The reagents and wash conditions are dependent on the nature of the second probe. For example, heat can be used with second probes which are polynucleotides, while detergents can be used with second probes that are antibodies and the like. The number of washes performed can be empirically determined, but typically range from 1× to 10×. If the pathogen biomolecules are bound by second probes under stringent or very stringent conditions, minimal to no washes may be utilized. Alternatively, if the pathogen biomolecules are bound by second probes under non-stringent conditions more washes may be needed. Optional drying steps may be used prior to addition of an enzyme at process block 212.

At process block 212, an enzyme substrate is introduced to the electrodes comprising the electrode probe-target pathogen biomolecule-second probe/enzyme. Choice of enzyme substrate is mostly dependent on the identity of the enzyme that is used in process block 208. For example, if the enzyme is HRP then TMB (3,3′,5,5′-Tetramethylbenzidine) may be used as the substrate. TMB acts as a hydrogen donor for the reduction hydrogen peroxide to water in the presence of HRP. Similarly, other common and commercially available substrates for HRP can also be used, as well as other substrates for the other oxidase enzymes described herein. The enzyme substrate is introduced under conditions that promote the conversion of the enzyme substrate to an oxidized produce by the action of the bound enzyme.

At process block 214, an electrochemical signal is generated from reducing the oxidized product using a bias potential applied to the electrodes of the sensor array. The redox cycle results in the shuttling of electrons by the substrate from the electrode to the HRP, producing enzymatic signal amplification of the current flow of the electrode. The concentration of the target pathogen biomolecule captured on the electrode surface can be quantified by the reduction current measured through the redox reaction between the substrate and enzyme by use of a potentiostat.

At decision block 216, the electrochemical signal generated from an electrode or electrodes of the sensor array, are correlated to the probe(s) bound to the electrode. As the probe is specific to pathogen biomolecules from a specific species of pathogen, the pathogen can be identified. If electrochemical signal is generated to multiple probes likely indicates multiple species of pathogens are present in the raw specimen. The identification results are outputted on a graphics user interface, and/or a printer. The graphics user interface can be connected to an automation system that is used to carry out one or more of the Process blocks of 100 to 214. The graphics user interface may be directly or remotely connected to the automation system.

Concurrently, sequentially, or prior to target pathogen(s) identification 200, target pathogen AST 300 can be carried out. Advantageously, target pathogen AST 300 can be carried out concurrently with target pathogen(s) identification 200, thereby providing AST results around the time in which the target pathogen(s) identification 200 results are reported. Alternatively, target pathogen AST 300

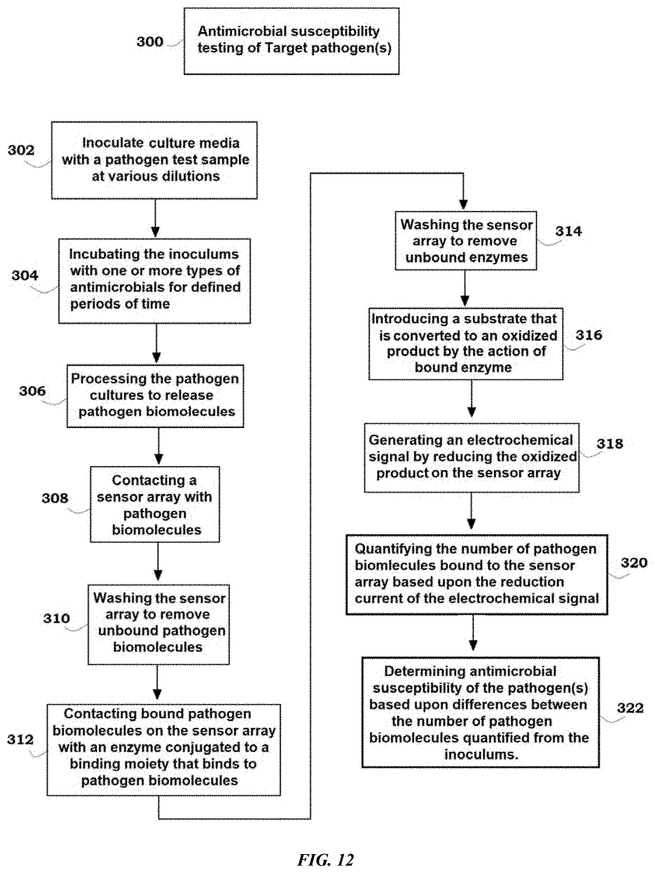

FIG. 12 presents a process flow for an example of antimicrobial susceptibility testing of pathogens 300. At process block 302, pathogen test sample 106 is used to inoculate a series of wells (e.g., strip wells) containing culture media. Various dilution factors of the inoculum can be used to inoculate the culture medium. Examples of dilution factors include, but are not limited to, 1×, 0.8×, 0.75×, 0.7×, 0.6×, 0.5×, 0.4×, 0.3×, 0.25×, 0.2×, 0.15×, 0.1×, 0.05×, 0.04×, 0.03×, 0.02×, 0.01×. Reasons for this dilution operation can include, but are not limited to, high starting concentration of target pathogen in the raw sample, overgrowth during the pathogen identification phase, and predicated concentration over 5E5 CFU/mL. In a particular embodiment, the culture medium is used to replicate the medium where the raw specimen was obtained from, e.g., urine, blood, saliva, etc. In another embodiment, the culture medium used in process block 302 is the same as the culture medium used in viability culture 104.

At process block 304, all or a portion of the inoculated cultures are incubated with various types of antimicrobials for a defined period of time (AST culture samples). Various concentrations of the antimicrobials may also be used with the inoculated cultures. One or more of the inoculated cultures are not incubated with antimicrobials. These cultures are used as control AST samples (growth control) to provide a baseline of pathogen growth and biomolecule production in pathogens in the absence of antimicrobials. One or more the AST culture samples may be combined or, alternatively, the AST cultures may be kept separate. AST cultures are cultured such that the concentration of the pathogen in the AST culture sample would increase in the absence of the identified antimicrobials. The culturing of the AST culture samples is performed for a defined period of time. The defined period of time can be 15 min, 30 min, 45 min, 60 min, 90 min, 120 min, 180 min, 240 min, 300 min, or a range that includes or is between any two of the foregoing time points. In some instances, the period of time the AST culture samples are cultured is a function of the pathogen identified, antimicrobial to be tested, and/or starting concentration of the initial inoculum. As noted above, more than one AST culture samples can include the same antimicrobial at different concentrations. As a result, each of the different cultures can be performed on an AST culture sample having a different concentration of the antimicrobial. Accordingly, the susceptibility of a pathogen to different concentrations of an antimicrobial can be tested. Examples of antimicrobials include, but are not limited to, antivirals, antifungals, antimycotic agents and antibiotics. Suitable antibiotics include, but are not limited to, penicillins (e.g., penicillin V potassium, amoxicillin, amoxicillin/clavulanate (Augmentin)); tetracyclines (e.g., doxycycline, tetracycline, minocycline); cephalosporins (e.g., cefuroxime, ceftriaxone, cefdinir); quinolones (e.g., ciprofloxacin, levofloxacin, moxifloxacin); lincomycins (e.g., clindamycin, lincomycin); macrolides (e.g., azithromycin, clarithromycin, erythromycin); sulfonamides (e.g., sulfamethoxazole-trimethoprim, sulfasalazine, sulfisoxazole); glycopeptide antibiotics (e.g., dalbavancin, oritavancin, telavancin, vancomycin); aminoglycosides (e.g., gentamicin, tobramycin, amikacin); and carbapenems (e.g., imipenem/cilastatin, meropenem, doripenem, ertapenem). Suitable antifungal agents include, but are not limited to, azole antifungals (e.g., itraconazole, posaconazole, ketoconazole, clotrimazole, miconazole, voriconazole); echinocandins (e.g., caspofungin, anidulafungin, micafungin); polyenes (e.g., nystatin, amphotericin b); and antimycotic agents (e.g., griseofulvin, terbinafine, flucytosine). In a particular embodiment, a method of the disclosure provides for use of one or more antimicrobials selected from Amikacin, Gentamicin, Kanamycin, Neomycin, Netilmicin, Tobramycin, Paromomycin, Streptomycin, Spectinomycin, Geldanamycin, Herbimycin, Rifaximin, Loracarbef, Ertapenem, Doripenem, Imipenem/Cilastatin, Meropenem, Cefadroxil, Cefazolin, Cephradine, Cephapirin, Cephalothin, Cefalexin, Cefaclor, Cefoxitin, Cefotetan, Cefamandole, Cefmetazole, Cefonicid, Loracarbef, Cefprozil, Cefuroxime, Cefixime, Cefdinir, Cefditoren, Cefoperazone, Cefotaxime, Cefpodoxime, Ceftazidime, Ceftibuten, Ceftizoxime, Moxalactam, Ceftriaxone, Cefepime, Ceftaroline fosamil, Ceftobiprole, Teicoplanin, Vancomycin, Telavancin, Dalbavancin, Oritavancin, Clindamycin, Lincomycin, Daptomycin, Azithromycin, Clarithromycin, Roxithromycin, Telithromycin, Spiramycin, Aztreonam, Furazolidone, Nitrofurantoin, Linezolid, Posizolid, Radezolid, Torezolid, Amoxicillin, Azlocillin, Dicloxacillin, Flucloxacillin, Mezlocillin, Methicillin, Nafcillin, Oxacillin, Penicillin G, Penicillin V, Piperacillin, Penicillin G, Temocillin, Ticarcillin, Amoxicillin/clavulanate, Ampicillin/sulbactam, Piperacillin/tazobactam, Ticarcillin/clavulanate, Bacitracin, Colistin, Polymyxin B, Ciprofloxacin, Enoxacin, Gatifloxacin, Gemifloxacin, Levofloxacin, Lomefloxacin, Moxifloxacin, Nadifloxacin, Nalidixic acid, Norfloxacin, Ofloxacin, Trovafloxacin, Grepafloxacin, Sparfloxacin, Temafloxacin, Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole, Sulfonamidochrysoidine, Demeclocycline, Doxycycline, Metacycline, Minocycline, Oxytetracycline, Tetracycline, Clofazimine, Dapsone, Capreomycin, Cycloserine, Ethambutol, Ethionamide, Isoniazid, Pyrazinamide, Rifampicin, Rifabutin, Rifapentine, Streptomycin, Arsphenamine, Chloramphenicol, Fosfomycin, Fusidic acid, Metronidazole, Mupirocin, Platensimycin, Quinupristin/Dalfopristin, Thiamphenicol, Tigecycline, Tinidazole, Trimethoprim, itraconazole, posaconazole, ketoconazole, clotrimazole, miconazole, voriconazole, caspofungin, anidulafungin, micafungin, nystatin, amphotericin b, griseofulvin, terbinafine, and/or flucytosine.

At process block 304, the AST culture samples are treated, typically with lysis buffer, sonication, Dounce homogenization, or the like to disrupt the pathogen cell wall and/or membranes to release biomolecules, including but not limited to, nucleic acids (e.g., RNA, DNA), proteins, lipids, carbohydrates, or combinations of any of the foregoing. A typical protocol can include addition of cell lysis buffer, and other reagents, such as inhibitors to proteases, RNases and/or DNases; buffers; stabilization agents; etc. In a particular embodiment, the pathogen test sample is processed to isolate or recover target pathogen RNA. The pathogen biomolecules are then contacted to electrodes of a sensor array. Probes are attached to the electrodes that are complementary and can bind to pathogen biomolecules. The probes can be any number of typical capture ligands, including oligo- or polynucleotides; antibodies; receptor ligands; receptors; aptamers, nucleotide binding proteins; etc. The surface of the electrode that is bound to the probe typically comprises a highly conductive material, like gold. The electrodes of the sensor array are typically spaced from each other so as to avoid cross contamination. The pathogen biomolecules can be manually dispensed to the electrodes of the sensory array, or can be dispensed using automated liquid handlers, such as liquid handlers of the UtiMax or BsiMax robotic systems. The pathogen biomolecules are then bound to the probes under conditions that promote binding of the probe to the pathogen biomolecules, or vice versa. To minimize nonspecific binding of pathogen biomolecules to the probes then increasingly stringent conditions can be used. For example, if the probes are oligonucleotides or polynucleotides and the pathogen biomolecules are RNA then stringent hybridization conditions can be used. In a particular embodiment, the probes bound to the electrodes of the sensor array are oligonucleotides or polynucleotides comprise sequences that bind to pathogen nucleic acids.

At process block 308, unbound or weakly bound pathogen biomolecules are removed from the electrodes by using a washing process. The stringency of the washing conditions can be used to remove all but the most strongly bound pathogen biomolecules (i.e., the most complementary to the probe). The reagents and wash conditions are dependent on the pathogen biomolecules being captures. For example, heat can be used with polynucleotides, while detergents can be used with proteins. The number of washes performed can be empirically determined, but typically range from 1× to 10×. If the pathogen biomolecules are bound to the probes under stringent or very stringent conditions, minimal to no washes may be utilized. Alternatively, if the pathogen biomolecules are bound to the probes under non-stringent conditions more washes may be needed. Optional drying steps may be used prior to addition of an enzyme at process block 310.

At process block 310, an enzyme in solution is then dispensed to each electrode of the sensor array. The enzyme used is typically an enzyme which can oxidize a substrate to make a product. Examples of such enzymes, include but are not limited to, horse radish peroxidase (HRP), glucose oxidase, xanthine oxidase, galactose oxidase, and lactate oxidase. The type and nature of the substrate is dependent on the substrate suitability for the enzyme used in the process. The enzyme disclosed herein is typical conjugated to a second probe that can bind to target pathogen biomolecules. Accordingly, pathogen biomolecules bound to probes attached to the electrodes are further bound by the second probe (i.e., the pathogen biomolecules are sandwiched between the two probes). The second probe can be any number of typical ligands, including oligo- or polynucleotides; antibodies; receptor ligands; receptors; aptamers; nucleotide binding proteins; etc. Choice of the second probe is largely dependent on the identity of the target biomolecule bound to the probes of the electrodes. For example, the second probe can be an antibody if the target biomolecule is a protein; or, alternatively, be a nucleotide sequence that hybridizes to pathogen nucleotides. In a further embodiment, the second probe is conjugated to HRP.

At process block 312, unbound or weakly bound second probes bound to pathogen biomolecules are removed using a washing process. The stringency of the washing conditions can be used to remove all but the most strongly bound second probes to pathogen biomolecules (i.e., the most complementary to the probe). The reagents and wash conditions are dependent on the nature of the second probe. For example, heat can be used with second probes which are polynucleotides, while detergents can be used with second probes that are antibodies and the like. The number of washes performed can be empirically determined, but typically range from 1× to 10×. If the pathogen biomolecules are bound by second probes under stringent or very stringent conditions, minimal to no washes may be utilized. Alternatively, if the pathogen biomolecules are bound by second probes under non-stringent conditions more washes may be needed. Optional drying steps may be used prior to addition of an substrate at process block 314.

At process block 314, an enzyme substrate is introduced to the electrodes comprising the electrode probe-target pathogen biomolecule-second probe/enzyme. Choice of enzyme substrate is mostly dependent on the identity of the enzyme that is used in process block 310. For example, if the enzyme is HRP then TMB (3,3′,5,5′-Tetramethylbenzidine) may be used as the substrate. TMB acts as a hydrogen donor for the reduction hydrogen peroxide to water in the presence of HRP. Similarly, other common and commercially available substrates for HRP can also be used, as well as other substrates for the other oxidase enzymes described herein. The enzyme substrate is introduced under conditions that promote the conversion of the enzyme substrate to an oxidized produce by the action of the bound enzyme.

At process block 316, an electrochemical signal is generated from reducing the oxidized product using a bias potential applied to the electrodes of the sensor array. The redox cycle results in the shuttling of electrons by the substrate from the electrode to the HRP, producing enzymatic signal amplification of the current flow of the electrode.

At process block 318, the concentration of the target pathogen biomolecule captured on the electrode surface can be quantified by the reduction current measured through the redox reaction between the substrate and enzyme by use of a potentiostat. The signal generated from voltammetry of each assay can indicate concentration levels and can serve as a growth indication factor. Accordingly, the voltammetry signal levels can be compared in order to effectively compare concentration levels. From which, the growth control (GC) ratio can be determined. In some instances, concentration is not used to measure growth.

At decision block 320, there is a strong correlation of reduction of growth control (GC) ratio while increasing antimicrobial concentrations is indicative of antimicrobial susceptibility. The minimum inhibitory concentration (MIC) of the pathogen to the antimicrobial can be calculated based on changes in the GC ratio. Changes in the GC ratio for the specific antimicrobials can be determined by measured differences in the amount of pathogen biomolecules bound to the electrodes when using different antimicrobials, or the same antimicrobial at different dilutions, in comparison to control AST samples that lack antimicrobials.

For instance, the GC ratio can be configured to show growth when the concentration of pathogen in an AST assay sample relative to the control concentration shows an increase of more than 50%, 100%, or 200% and/or when a factor that indicates a concentration level or is a function of the concentration level indicates an increase of more than 50%, 100%, or 200%.

When the AST sample is indicated as being positive (classified as showing pathogen growth) for a particular AST assay sample, the pathogen is reported as being resistant to the concentration of the antimicrobial in the AST culture sample (decision block 320). When the determination at decision block 320 is negative (classified as not showing pathogen growth) for a particular AST assay sample, the pathogen is reported as being susceptible to the concentration of the antimicrobial in the AST culture sample (decision block 320). The results for at least each of the AST assay samples that includes one or more of the identified antimicrobials can be reported. Other classifications are possible in addition to positive and negative. For instance, an AST assay sample can be classified as showing intermediate growth. The AST results are outputted on a graphics user interface, and/or a printer. The graphics user interface can be connected to an automation system that is used to carry out one or more of blocks of 100 to 320. The graphics user interface may be directly or remotely connected to the automation system.

The duration of the susceptibility cultures is measured from the start of the culture at process block 304. The susceptibility culture time is considered to be zero minutes at the start of the susceptibility culture. In general, a susceptibility culture is considered to be started when the AST culture sample is heated. Accordingly, when a robotic system such as the UtiMax or BsiMax is used to conduct the susceptibility culture, the susceptibility culture is considered to be started when the UtiMax or BsiMax starts to heat the sample.

The assays (growth tests) in the antimicrobial susceptibility testing phase can be performed using the same assays as were used in the pathogen characterization phase. For instance, the growth tests in the antimicrobial susceptibility testing phase be performed manually or using a robotic system such as the UtiMax or BsiMax from GeneFluidics, Inc. of Irwindale, Ca., USA. Additionally, the reagents used in these assays can be purchased in reagent kits such as the UtiMax and the BsiMax reagent kit from GeneFluidics, Inc. of Irwindale, Ca., USA. The “16×PID/AST Sensor Chips” are electrochemical sensors sold by GeneFluidics, Inc. of Irwindale, Ca., USA for manual or automated use with the UtiMax and the BsiMax reagent kits. As noted above, these growth tests can be performed on site.

When using a robotic system such as the UtiMax or BsiMax, the testing time period from a user receiving or system generating the AST test sample to a user receiving the results of the assay(s) (indication of the growth of pathogen in an AST assay sample) is generated is generally on the order of 120 minutes for a raw sample of urine and 120 minutes for a raw sample of whole blood. When manually performing an assay, the testing time period from a user receiving the AST test sample to a user receiving the results of the assay(s) (indication of the growth of pathogen in an AST assay sample) is generated is generally on the order of 120 minutes for urine and 120 minutes for whole blood. Accordingly, the testing time period can be greater than 20 minutes, 60 minutes or 90 minutes and/or less than 360 minutes, 180 minutes or 45 minutes.

The results of the Antimicrobial Susceptibility Testing (AST) phase are available in a time period that is approximately equal to the sum of the time to prepare the AST culture samples, the AST culture time, and the testing time period. Accordingly, the results of the antimicrobial susceptibility testing phase can be received in a time period greater than 45 minutes, 120 minutes and/or less than 5 hours or 3 hours from a user of a robotic system or the robotic system receiving the one or more test samples from which the Initial AST sample(s) is prepared.

A prescription can be made using one, two, or three factors selected from the group consisting the pathogen identity, pathogen concentration in the raw sample, antimicrobial resistance, or antimicrobial susceptibility. In an example where all three factors are used, guidelines that associate one or more particular antimicrobial medications with particular pathogens can be used to identify one or more antimicrobials that can be used to treat the identified pathogen(s). The one or more identified antimicrobials that are reported as susceptible can then be selected for a prescription. This prescription process avoids the prescription of antimicrobials to which the identified pathogen is resistant and accordingly reduces the creation of additional resistance. Additionally, using the concentration of pathogen in the raw sample as a factor in determining dosage can reduce overdosing and/or underdosing and can further reduces the creation of additional resistance.

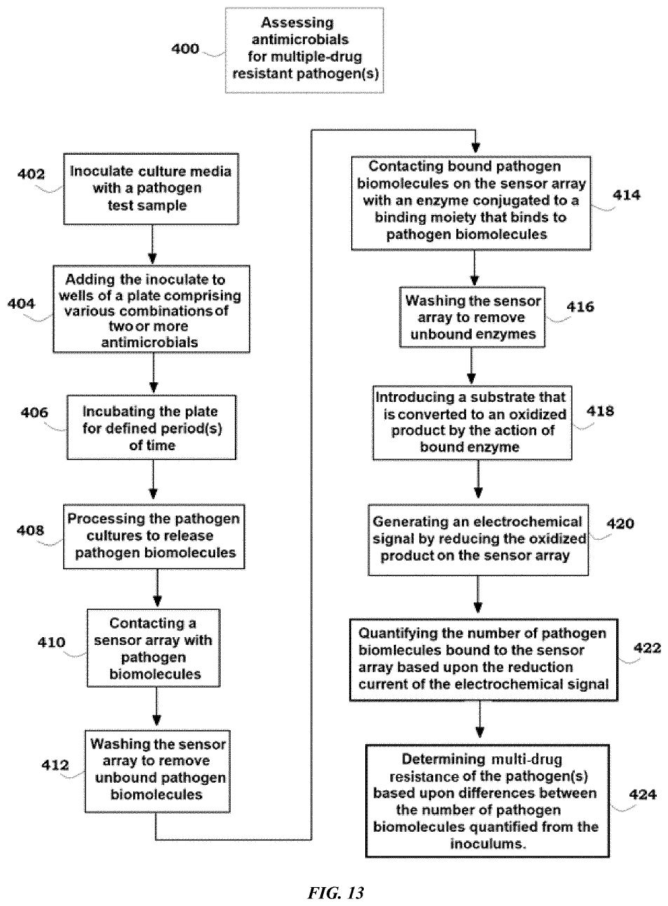

FIG. 13 presents a process flow for an example of multi-drug resistance testing of pathogens 400. At process block 402, pathogen test sample 106 is used to inoculate container(s) containing culture media. Various dilution factors of the inoculum can be used to inoculate the culture medium. Examples of dilution factors include, but are not limited to, 1×, 0.8×, 0.75×, 0.7×, 0.6×, 0.5×, 0.4×, 0.3×, 0.25×, 0.2×, 0.15×, 0.1×, 0.05×, 0.04×, 0.03×, 0.02×, 0.01×. Reasons for this dilution operation can include, but are not limited to, high starting concentration of target pathogen in the raw sample, overgrowth during the pathogen identification phase, and predicated concentration over 5E5 CFU/mL. In a particular embodiment, the culture medium is used to replicate the medium where the raw specimen was obtained from, e.g., urine, blood, saliva, etc. In another embodiment, the culture medium used in process block 402 is the same as the culture medium used in viability culture 104.

At process block 404, the inoculate is added to the wells of a multi-well plate (e.g., a 6-well plate, a 12-well plate, a 24-well plate, a 96-well plate, a 386-well plate) containing various combinations of two or more antimicrobials in cell culture media. The concentration of the pathogens added to each well of the multi-well plate can be normalized so that a certain number of pathogens are evenly added to each well of the multi-well plate. In another embodiment, the culture medium used in process block 404 is the same as the culture medium used in process block 402. The combinations of antibiotics are determined prior and are generally selected for possible additive or synergistic action for targeted pathogens. The combinations of antimicrobials comprise combinations of the same antimicrobials but used at different concentrations or ratios. The combinations of antimicrobials can also comprise combinations of different types or classes of antibiotics. Typically, the combinations of antimicrobials refer to combinations of 2 or 3 antimicrobials, but may include 4 or more antimicrobials.

At process block 406, all or a portion of the inoculated cultures are incubated with various types of antimicrobials for a defined period of time (AST culture samples). Various concentrations of a combination of antimicrobials may also be used with the inoculated cultures. One or more of the inoculated cultures are not incubated with antimicrobials. These cultures are used as control samples (growth control) to provide a baseline of pathogen growth and biomolecule production in pathogens in the absence of antimicrobials. One or more the AST culture samples may be combined or, alternatively, the AST cultures may be kept separate. AST cultures are cultured such that the concentration of the pathogen in the AST culture sample would increase in the absence of the identified antimicrobials. The culturing of the AST culture samples is performed for a defined period of time. The defined period of time can be 15 min, 30 min, 45 min, 60 min, 90 min, 120 min, 180 min, 240 min, 300 min, or a range that includes or is between any two of the foregoing time points. In some instances, the period of time the AST culture samples are cultured is a function of the pathogen identified, antimicrobial to be tested, and/or starting concentration of the initial inoculum. As noted above, more than one AST culture samples can include the same antimicrobial at different concentrations. As a result, each of the different cultures can be performed on an AST culture sample having a different concentration of the antimicrobial. Accordingly, the susceptibility of a pathogen to different concentrations of an antimicrobial can be tested.

At process block 408, the pathogen culture samples are treated, typically with lysis buffer, sonication, Dounce homogenization, or the like to disrupt the pathogen cell wall and/or membranes to release biomolecules, including but not limited to, nucleic acids (e.g., RNA, DNA), proteins, lipids, carbohydrates, or combinations of any of the foregoing. A typical protocol can include addition of cell lysis buffer, and other reagents, such as inhibitors to proteases, RNases and/or DNases; buffers; stabilization agents; etc. In a particular embodiment, the pathogen test sample is processed to isolate or recover target pathogen RNA.

At process block 410, the pathogen biomolecules are then contacted to electrodes of a sensor array. Probes are attached to the electrodes that are complementary and can bind to pathogen biomolecules. The probes can be any number of typical capture ligands, including oligo- or polynucleotides; antibodies; receptor ligands; receptors; aptamers, nucleotide binding proteins; etc. The surface of the electrode that is bound to the probe typically comprises a highly conductive material, like gold. The electrodes of the sensor array are typically spaced from each other so as to avoid cross contamination. The pathogen biomolecules can be manually dispensed to the electrodes of the sensory array, or can be dispensed using automated liquid handlers, such as liquid handlers of the UtiMax or BsiMax robotic systems. The pathogen biomolecules are then bound to the probes under conditions that promote binding of the probe to the pathogen biomolecules, or vice versa. To minimize nonspecific binding of pathogen biomolecules to the probes then increasingly stringent conditions can be used. For example, if the probes are oligonucleotides or polynucleotides and the pathogen biomolecules are RNA then stringent hybridization conditions can be used. In a particular embodiment, the probes bound to the electrodes of the sensor array are oligonucleotides or polynucleotides comprise sequences that bind to pathogen nucleic acids.

At process block 412, unbound or weakly bound pathogen biomolecules are removed from the electrodes by using a washing process. The stringency of the washing conditions can be used to remove all but the most strongly bound pathogen biomolecules (i.e., the most complementary to the probe). The reagents and wash conditions are dependent on the pathogen biomolecules being captures. For example, heat can be used with polynucleotides, while detergents can be used with proteins. The number of washes performed can be empirically determined, but typically range from 1× to 10×. If the pathogen biomolecules are bound to the probes under stringent or very stringent conditions, minimal to no washes may be utilized. Alternatively, if the pathogen biomolecules are bound to the probes under non-stringent conditions more washes may be needed. Optional drying steps may be used prior to addition of an enzyme at process block 414.

At process block 414, an enzyme in solution is then dispensed to each electrode of the sensor array. The enzyme used is typically an enzyme which can oxidize a substrate to make a product. Examples of such enzymes, include but are not limited to, horse radish peroxidase (HRP), glucose oxidase, xanthine oxidase, galactose oxidase, and lactate oxidase. The type and nature of the substrate is dependent on the substrate suitability for the enzyme used in the process. The enzyme disclosed herein is typical conjugated to a second probe that can bind to target pathogen biomolecules. Accordingly, pathogen biomolecules bound to probes attached to the electrodes are further bound by the second probe (i.e., the pathogen biomolecules are sandwiched between the two probes). The second probe can be any number of typical ligands, including oligo- or polynucleotides; antibodies; receptor ligands; receptors; aptamers; nucleotide binding proteins; etc. Choice of the second probe is largely dependent on the identity of the target biomolecule bound to the probes of the electrodes. For example, the second probe can be an antibody if the target biomolecule is a protein; or, alternatively, be a nucleotide sequence that hybridizes to pathogen nucleotides. In a further embodiment, the second probe is conjugated to HRP.

At process block 416, unbound or weakly bound second probes bound to pathogen biomolecules are removed using a washing process. The stringency of the washing conditions can be used to remove all but the most strongly bound second probes to pathogen biomolecules (i.e., the most complementary to the probe). The reagents and wash conditions are dependent on the nature of the second probe. For example, heat can be used with second probes which are polynucleotides, while detergents can be used with second probes that are antibodies and the like. The number of washes performed can be empirically determined, but typically range from 1× to 10×. If the pathogen biomolecules are bound by second probes under stringent or very stringent conditions, minimal to no washes may be utilized. Alternatively, if the pathogen biomolecules are bound by second probes under non-stringent conditions more washes may be needed. Optional drying steps may be used prior to addition of a substrate at process block 418.

At process block 418, an enzyme substrate is introduced to the electrodes comprising the electrode probe-target pathogen biomolecule-second probe/enzyme. Choice of enzyme substrate is mostly dependent on the identity of the enzyme that is used in process block 414. For example, if the enzyme is HRP then TMB (3,3′,5,5′-Tetramethylbenzidine) may be used as the substrate. TMB acts as a hydrogen donor for the reduction hydrogen peroxide to water in the presence of HRP. Similarly, other common and commercially available substrates for HRP can also be used, as well as other substrates for the other oxidase enzymes described herein. The enzyme substrate is introduced under conditions that promote the conversion of the enzyme substrate to an oxidized produce by the action of the bound enzyme.

At process block 420, an electrochemical signal is generated from reducing the oxidized product using a bias potential applied to the electrodes of the sensor array. The redox cycle results in the shuttling of electrons by the substrate from the electrode to the HRP, producing enzymatic signal amplification of the current flow of the electrode.

At process block 422, the concentration of the target pathogen biomolecule captured on the electrode surface can be quantified by the reduction current measured through the redox reaction between the substrate and enzyme by use of a potentiostat. The signal generated from voltammetry of each assay can indicate concentration levels and can serve as a growth indication factor. Accordingly, the voltammetry signal levels can be compared in order to effectively compare concentration levels. From which, the growth control (GC) ratio can be determined. In some instances, concentration is not used to measure growth.

At decision block 424, there is a strong correlation of reduction of a growth control (GC) ratio when a pathogen is susceptible to a combination of antimicrobials. The greater the reduction of the GC ratio the more susceptible the pathogen is to the particular combination of antimicrobials. Based upon overlapping combinations, the most effective combination of antimicrobials and/or the most effective dose of the antimicrobials, can be identified for the targeted pathogens. Further, the minimum inhibitory concentration (MIC) of the pathogen to the combination of antimicrobials can be calculated based on changes in the GC ratio. Changes in the GC ratio for the specific combination of antimicrobials can be determined by measured differences in the amount of pathogen biomolecules bound to the electrodes when using different combinations of antimicrobials, or the same combination of antimicrobials at different dilutions, in comparison to control samples that lack antimicrobials.

For instance, the GC ratio can be configured to show growth when the concentration of pathogen in an assay sample relative to the control concentration shows an increase of more than 50%, 100%, or 200% and/or when a factor that indicates a concentration level or is a function of the concentration level indicates an increase of more than 50%, 100%, or 200%.

When the sample is indicated as being positive (classified as showing pathogen growth) for a particular culture sample, the pathogen is reported as being multi-drug resistant to the particular combination of antimicrobials (decision block 424). When the determination at decision block 424 is negative (classified as not showing pathogen growth) for a particular culture sample, the pathogen is reported as being susceptible to the particular combinations of antimicrobials in the culture sample (decision block 424). The results for at least each of the culture samples demonstrating susceptibility for the combinations of antimicrobials can then be reported. Other classifications are possible in addition to positive and negative. For instance, a culture sample can be classified as showing intermediate growth. The MDR results are outputted on a graphics user interface, and/or a printer. The graphics user interface can be connected to an automation system that is used to carry out one or more of blocks of 100 to 424. The graphics user interface may be directly or remotely connected to the automation system.

The duration of the susceptibility cultures is measured from the start of the culture at process block 406. The susceptibility culture time is considered to be zero minutes at the start of the susceptibility culture. In general, a susceptibility culture is considered to be started when the culture sample is heated. Accordingly, when a robotic system such as the UtiMax or BsiMax is used to conduct the susceptibility culture, the susceptibility culture is considered to be started when the UtiMax or BsiMax starts to heat the sample.

The assays (growth tests) in the antimicrobial susceptibility testing phase can be performed using the same assays as were used in the pathogen characterization phase. For instance, the growth tests in the antimicrobial susceptibility testing phase be performed manually or using a robotic system such as the UtiMax or BsiMax from GeneFluidics, Inc. of Irwindale, Ca., USA. Additionally, the reagents used in these assays can be purchased in reagent kits such as the UtiMax and the BsiMax reagent kit from GeneFluidics, Inc. of Irwindale, Ca., USA. The “16×PID/AST Sensor Chips” are electrochemical sensors sold by GeneFluidics, Inc. of Irwindale, Ca., USA for manual or automated use with the UtiMax and the BsiMax reagent kits. As noted above, these growth tests can be performed on site.

When using a robotic system such as the UtiMax or BsiMax, the testing time period from a user receiving or system generating the AST test sample to a user receiving the results of the assay(s) (indication of the growth of pathogen in an AST assay sample) is generated is generally on the order of 120 minutes for a raw sample of urine and 120 minutes for a raw sample of whole blood. When manually performing an assay, the testing time period from a user receiving the AST test sample to a user receiving the results of the assay(s) (indication of the growth of pathogen in an AST assay sample) is generated is generally on the order of 120 minutes for urine and 120 minutes for whole blood. Accordingly, the testing time period can be greater than 20 minutes, 60 minutes or 90 minutes and/or less than 360 minutes, 180 minutes or 45 minutes.

The results of the MDR testing are available in a time period that is approximately equal to the sum of the time to prepare the culture samples, the culture time, and the testing time period. Accordingly, the results of the antimicrobial susceptibility testing phase can be received in a time period greater than 45 minutes, 120 minutes and/or less than 5 hours or 3 hours from a user of a robotic system or the robotic system receiving the one or more test samples from which the Initial AST sample(s) is prepared.

A prescription can be made using one, two, or three factors selected from the group consisting the pathogen identity, pathogen concentration in the raw sample, antimicrobial resistance, or antimicrobial susceptibility. In an example where all three factors are used, guidelines that associate one or more particular antimicrobial medications with particular pathogens can be used to identify a combination of antimicrobials that can be used to treat the identified pathogen(s). The one or more identified antimicrobials that are reported as susceptible can then be selected for a prescription. This prescription process avoids the prescription of antimicrobials to which the identified pathogen is resistant and accordingly reduces the creation of additional resistance. Additionally, using the concentration of pathogen in the raw sample as a factor in determining dosage can reduce overdosing and/or underdosing and can further reduces the creation of additional resistance.

The assays disclosed above in the various process blocks be performed manually. Alternately, as disclosed above, the assays in the above process blocks can be performed using a robotic system such as the UtiMax or BsiMaxUtiMax or BsiMax from GeneFluidics, Inc. of Irwindale, Ca., USA. A suitable electrochemical sensor for use in performing the above assays is the UtiMax sensor chip and/or the BsiMax ID/AST sensor chip sold by GeneFluidics, Inc. of Irwindale, Ca., USA. The assays can be performed by using these electrochemical sensors manually or with one of the robotics systems such as the UtiMax or BsiMaxUtiMax or BsiMax Robotic System and/or the Lab Automation System sold by GeneFluidics, Inc. of Irwindale, Ca., USA. The reagents used in these assays can be purchased in reagent kits. For instance, a reagent kit for use with urinary tract infections is the UtiMax sold by GeneFluidics, Inc. of Irwindale, Ca., USA. Pathogens that can be identified with the UtiMax reagent kit include E. coli, P. aeruginosa, and K. Pneumoniae and more depending on the ID sensor chip configuration. The Limit of Detection (LOD) for an assay performed using the UtiMax reagent kit in combination with the with the UtiMax or BsiMax system and the above method of preparing the assay sample has been shown to be 10,000 CFU/mL with zero culture time before taking the aliquoted portion of the viability sample. A reagent kit for use with bloodstream infections is the BsiMax sold by GeneFluidics, Inc. of Irwindale, Ca. Pathogens that can be identified with the BsiMax reagent kit include E. coli, P. aeruginosa, and S. aureus and more depending on the ID sensor chip configuration. The Limit of Detection (LOD) for an assay performed using the BsiMax reagent kit in combination with the UtiMax or BsiMax system and the above method of preparing the assay sample has been shown to be 4 (CFU/mL) with a 5-hour culture time before taking the aliquoted portion of the viability sample. These LOD numbers are determined according to Clinical and Laboratory Standards Institute (CLSI)I document EP17-A, “Evaluation of Detection Capability for Clinical Laboratory Measurement Procedures.” The UtiMax and the BsiMax reagent kits come with electrochemical sensors that can be used manually or with a robotic system such as the UtiMax or BsiMax from GeneFluidics, Inc. of Irwindale, Ca., USA.

The assay sample can be prepared manually from the raw sample and/or the assay sample can be manually assayed. Alternately, as disclosed above, a robotic system such as the UtiMax or BsiMax can be used to prepare the assay sample from the raw sample and/or to perform the assay of the assay sample

The low Limits of Detection (LOD) associated with the pathogen characterization phase described above also permits the antimicrobial susceptibility testing phase to be performed in a period of time that allows a prescription to be made that is both in a reasonable period of time after taking the raw sample and is based on antimicrobial susceptibility information. For instance, the raw sample can be cultured in the presence of each one of the antimicrobials that are identified as described above. A different culture can be performed for each of the identified antimicrobials. Additionally, one or more cultures can be performed for each of the identified antimicrobials. When more than one culture is performed for a single antimicrobial, the different cultures can be performed with different concentrations of antimicrobial.

In a particular embodiment, the disclosure further provides the following aspects of methods and systems of the disclosure (Aspect 1 to 14 as follows):

Aspect 1. A fully automated process using a robotic handling system with a microorganism test sample comprising unknown or unidentified microorganism(s), wherein the process comprises one or more of the following steps:

identifying unknown or unidentified microorganism(s) in the microorganism test sample by using a sensor chip comprising an array of electrodes, wherein each electrode comprises a probe that is specific to an 16S rRNA from a particular microorganism species or to a group of microorganisms, wherein the identity of the unknown or unidentified microorganism(s) in the microorganism(s) sample can be determined based upon an electrochemical signal generated when a probe hybridizes to complementary molecules from the unknown or unidentified microorganism under stringent reaction conditions; and/or

assessing antimicrobial susceptibility of the microorganism(s) in the microorganism test sample by using a sensor chip comprising an array of electrodes, wherein each electrode is used to measure a reduction current that correlates to the growth control (GC) ratio of the microorganism(s), wherein the microorganism(s) are exposed to a panel of antimicrobials, and wherein antimicrobial susceptibly of the microorganism can be assessed based upon the downward trend of the GC ratio to increasing concentrations and/or exposure to the antimicrobial, by measuring a decrease in the reduction current at the electrodes; and/or

assessing multidrug resistance of the microorganism(s) in the microorganism test sample by using a sensor chip comprising an array of electrodes, wherein each electrode is used to measure a reduction current that correlates to the growth control (GC) ratio of the microorganism(s), wherein the microorganism(s) are exposed to combinations of antimicrobials used at various concentrations, and wherein multidrug resistance of the microorganism(s) can be assessed based upon a reduction of the GC ratio when exposed to specific combinations of antimicrobials, by measuring a decrease in the reduction current at the electrodes;

wherein the microorganism test sample is generated directly from an unprocessed sample without the use of clinical isolates.

Aspect 2. The method of aspect 1, wherein the microorganism test sample is generated directly from an unprocessed sample without the use of clinical isolates comprising the steps of:

(i) loading an unprocessed sample into a processing module of a robotic handling system;

(ii) isolating or purifying microorganisms from the raw or unprocessed sample using a processing module of the robotic handling system;

(iii) generating a viability culture of the isolated or purified unidentified and/or unknown microorganisms by using a liquid handler module of the robotic system to add culture medium to the isolated or purified microorganisms and incubating the microorganisms from 30 minutes to 330 minutes; and

(iv) generating a microorganism test sample by isolating or purifying microorganisms from the viability culture by using the processing module of the robotic handling system.

Aspect 3. The method of aspect 1 or 2, wherein the unprocessed sample is a sample obtained from a subject, preferably a blood, urine, saliva, or cerebrospinal fluid (CSF) sample, more preferably a blood or urine sample.

Aspect 4. The method of any one of the preceding aspects, wherein the processing module of the robotic handling system is a centrifugation module.

Aspect 5. The method of any one of the preceding aspects, wherein the microorganisms are incubated from 10 minutes to 330 minutes, from 10 minutes to 300 minutes, from 10 minutes to 270 minutes, from 10 minutes to 240 minutes, from 10 minutes to 200 minutes, from 10 minutes to 180 minutes, from 10 minutes to 150 minutes, from 10 minutes to 120 minutes, from 10 minutes to 90 minutes, from 10 minutes to 60 minutes, from 10 minutes to 45 minutes, or from 10 minutes to 30 minutes, optionally at 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C. or a range that includes any two of the foregoing temperatures.

Aspect 6. The method of aspect 1, wherein the unknown or unidentified microorganism in the microorganism test sample is identified by the following steps:

adding lysis buffer to the microorganism test sample to release nucleic acids (e.g., RNA, DNA, etc.) and loading the lysates on the array of electrodes of the sensor chip using the liquid handler module of the robotics system, wherein the electrodes comprise a 16S rRNA probe immobilized on the surface of each of the electrodes, wherein each electrode comprises a 16S rRNA probe that hybridizes under stringent reaction conditions to the 16S rRNA from a specific microorganism species;

hybridizing the released nucleic acids to 16S rRNA probes by incubating the sensor chip under stringent reaction conditions;

stringently washing and drying the array of electrodes to remove any unbound nucleic acids or other matrix components;

dispending an enzyme reagent to the electrodes using a liquid handler module of the robotics system and hybridizing the enzyme reagent (e.g., horse radish peroxidase) to nucleic acids bound to the 16S rRNA probes;

stringently washing and drying the array of electrodes to remove any unbound enzyme reagent;

dispending an enzyme substrate (e.g., 3,3′,5,5′-tetramethylbenzidine (TMB)) to the electrodes using a liquid handler module of the robotics system;

transporting the sensor chip to a reader module of the robotic system, and performing an enzymatic amperometric reading to identify electrode(s) that produce a reduction current; and

identifying the microorganism(s) by correlating the electrodes that produce a reduction current with the identity of the immobilized 16S rRNA probe.

Aspect 7. The method of any one of aspects 1 to 6, wherein the unknown or unidentified microorganism in the microorganism test sample can be assessed for antimicrobial susceptibility according to the following:

inoculating culture media with the microorganism test sample at various dilutions using a liquid handler of the robotic system;

dispensing inoculums to wells (e.g., strip wells) comprising culture media and an antimicrobial, and to wells comprising just culture media, wherein the antimicrobial can be used at various dilutions and the wells can comprise a different antimicrobial;

incubating wells comprising the inoculums for 10 minutes to 330 minutes, from 10 minutes to 300 minutes, from 10 minutes to 270 minutes, from 10 minutes to 240 minutes, from 10 minutes to 200 minutes, from 10 minutes to 180 minutes, from 10 minutes to 150 minutes, from 10 minutes to 120 minutes, from 10 minutes to 90 minutes, from 10 minutes to 60 minutes, from 10 minutes to 45 minutes, or from 10 minutes to 30 minutes, optionally at 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C. or a range that includes any two of the foregoing temperatures;

adding lysis buffer to the inoculums to release nucleic acids (e.g., RNA, DNA, etc.) and loading the lysates on the array of electrodes of the sensor chip using the liquid handler module of the robotics system, wherein the electrodes comprise a probe immobilized on the surface of each of the electrodes that specifically binds to nucleic acids from microorganisms;

hybridizing the released nucleic acids to the probes by incubating the sensor chip under stringent reaction conditions;

stringently washing and drying the array of electrodes to remove any unbound nucleic acids or other matrix components;

dispending an enzyme reagent to the electrodes using a liquid handler module of the robotics system and hybridizing the enzyme reagent (e.g., horse radish peroxidase) to nucleic acids bound to the probes;

stringently washing and drying the array of electrodes to remove any unbound enzyme reagent;

dispending an enzyme substrate (e.g., 3,3′,5,5′-tetramethylbenzidine (TMB)) to the electrodes using a liquid handler module of the robotics system;

transporting the sensor chip to a reader module of the robotic system, and performing an enzymatic amperometric reading to identify electrode(s) that produce a reduction current; and

determining that the microorganism(s) are susceptible to antimicrobial(s) based upon a decreasing trend in the reduction current in response to increasing concentration of the antimicrobial(s).

Aspect 8. The method of aspect 7, wherein the antimicrobial(s) are selected from antivirals, antifungals, antimycotic agents and antibiotics.

Aspect 9. The method of claim 7, wherein the antimicrobial(s) are selected from Amikacin, Gentamicin, Kanamycin, Neomycin, Netilmicin, Tobramycin, Paromomycin, Streptomycin, Spectinomycin, Geldanamycin, Herbimycin, Rifaximin, Loracarbef, Ertapenem, Doripenem, Imipenem/Cilastatin, Meropenem, Cefadroxil, Cefazolin, Cephradine, Cephapirin, Cephalothin, Cefalexin, Cefaclor, Cefoxitin, Cefotetan, Cefamandole, Cefmetazole, Cefonicid, Loracarbef, Cefprozil, Cefuroxime, Cefixime, Cefdinir, Cefditoren, Cefoperazone, Cefotaxime, Cefpodoxime, Ceftazidime, Ceftibuten, Ceftizoxime, Moxalactam, Ceftriaxone, Cefepime, Ceftaroline fosamil, Ceftobiprole, Teicoplanin, Vancomycin, Telavancin, Dalbavancin, Oritavancin, Clindamycin, Lincomycin, Daptomycin, Azithromycin, Clarithromycin, Roxithromycin, Telithromycin, Spiramycin, Aztreonam, Furazolidone, Nitrofurantoin, Linezolid, Posizolid, Radezolid, Torezolid, Amoxicillin, Azlocillin, Dicloxacillin, Flucloxacillin, Mezlocillin, Methicillin, Nafcillin, Oxacillin, Penicillin G, Penicillin V, Piperacillin, Penicillin G, Temocillin, Ticarcillin, Amoxicillin/clavulanate, Ampicillin/sulbactam, Piperacillin/tazobactam, Ticarcillin/clavulanate, Bacitracin, Colistin, Polymyxin B, Ciprofloxacin, Enoxacin, Gatifloxacin, Gemifloxacin, Levofloxacin, Lomefloxacin, Moxifloxacin, Nadifloxacin, Nalidixic acid, Norfloxacin, Ofloxacin, Trovafloxacin, Grepafloxacin, Sparfloxacin, Temafloxacin, Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole, Sulfonamidochrysoidine, Demeclocycline, Doxycycline, Metacycline, Minocycline, Oxytetracycline, Tetracycline, Clofazimine, Dapsone, Capreomycin, Cycloserine, Ethambutol, Ethionamide, Isoniazid, Pyrazinamide, Rifampicin, Rifabutin, Rifapentine, Streptomycin, Arsphenamine, Chloramphenicol, Fosfomycin, Fusidic acid, Metronidazole, Mupirocin, Platensimycin, Quinupristin/Dalfopristin, Thiamphenicol, Tigecycline, Tinidazole, Trimethoprim, itraconazole, posaconazole, ketoconazole, clotrimazole, miconazole, voriconazole, caspofungin, anidulafungin, micafungin, nystatin, amphotericin b, griseofulvin, terbinafine, and flucytosine.

Aspect 10. The method of any one of aspects 1 to 9, wherein the unknown or unidentified microorganism in the microorganism test sample can be assessed for multidrug resistance (MDR) according to the following:

inoculating culture media with the microorganism test sample at various dilutions using a liquid handler of the robotic system;

dispensing inoculums to wells (e.g., wells of a 96 well plate) comprising culture media and various combinations of antimicrobials (e.g., 2, 3, 4, 5 or more antimicrobials), and to wells comprising just culture media, wherein the combinations of antimicrobials can include the same combination of antimicrobials, but used as different ratios or concentrations, or combination of different antimicrobials;

incubating wells comprising the inoculums for 10 minutes to 330 minutes, from 10 minutes to 300 minutes, from 10 minutes to 270 minutes, from 10 minutes to 240 minutes, from 10 minutes to 200 minutes, from 10 minutes to 180 minutes, from 10 minutes to 150 minutes, from 10 minutes to 120 minutes, from 10 minutes to 90 minutes, from 10 minutes to 60 minutes, from 10 minutes to 45 minutes, or from 10 minutes to 30 minutes, optionally at 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C. or a range that includes any two of the foregoing temperatures;

adding lysis buffer to the inoculums to release nucleic acids (e.g., RNA, DNA, etc.) and loading the lysates on the array of electrodes of the sensor chip using the liquid handler module of the robotics system, wherein the electrodes comprise a probe immobilized on the surface of each of the electrodes that specifically binds to nucleic acids from microorganisms;

hybridizing the released nucleic acids to the probes by incubating the sensor chip under stringent reaction conditions;

stringently washing and drying the array of electrodes to remove any unbound nucleic acids or other matrix components;

dispending an enzyme reagent to the electrodes using a liquid handler module of the robotics system and hybridizing the enzyme reagent (e.g., horse radish peroxidase) to nucleic acids bound to the probes;

stringently washing and drying the array of electrodes to remove any unbound enzyme reagent;

dispending an enzyme substrate (e.g., 3,3′,5,5′-tetramethylbenzidine (TMB)) to the electrodes using a liquid handler module of the robotics system;

transporting the sensor chip to a reader module of the robotic system, and performing an enzymatic amperometric reading to identify electrode(s) that produce a reduction current; and

determining the MDR of the microorganism(s) based upon changes in the reduction current in response to different combinations of antimicrobials, or the same combination of antimicrobials, but used at different dilutions.

Aspect 11. The method of aspect 10, wherein the antimicrobial(s) are selected from antivirals, antifungals, antimycotic agents and antibiotics.

Aspect 12. The method of claim 10, wherein the antimicrobial(s) are selected from Amikacin, Gentamicin, Kanamycin, Neomycin, Netilmicin, Tobramycin, Paromomycin, Streptomycin, Spectinomycin, Geldanamycin, Herbimycin, Rifaximin, Loracarbef, Ertapenem, Doripenem, Imipenem/Cilastatin, Meropenem, Cefadroxil, Cefazolin, Cephradine, Cephapirin, Cephalothin, Cefalexin, Cefaclor, Cefoxitin, Cefotetan, Cefamandole, Cefmetazole, Cefonicid, Loracarbef, Cefprozil, Cefuroxime, Cefixime, Cefdinir, Cefditoren, Cefoperazone, Cefotaxime, Cefpodoxime, Ceftazidime, Ceftibuten, Ceftizoxime, Moxalactam, Ceftriaxone, Cefepime, Ceftaroline fosamil, Ceftobiprole, Teicoplanin, Vancomycin, Telavancin, Dalbavancin, Oritavancin, Clindamycin, Lincomycin, Daptomycin, Azithromycin, Clarithromycin, Roxithromycin, Telithromycin, Spiramycin, Aztreonam, Furazolidone, Nitrofurantoin, Linezolid, Posizolid, Radezolid, Torezolid, Amoxicillin, Azlocillin, Dicloxacillin, Flucloxacillin, Mezlocillin, Methicillin, Nafcillin, Oxacillin, Penicillin G, Penicillin V, Piperacillin, Penicillin G, Temocillin, Ticarcillin, Amoxicillin/clavulanate, Ampicillin/sulbactam, Piperacillin/tazobactam, Ticarcillin/clavulanate, Bacitracin, Colistin, Polymyxin B, Ciprofloxacin, Enoxacin, Gatifloxacin, Gemifloxacin, Levofloxacin, Lomefloxacin, Moxifloxacin, Nadifloxacin, Nalidixic acid, Norfloxacin, Ofloxacin, Trovafloxacin, Grepafloxacin, Sparfloxacin, Temafloxacin, Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole, Sulfonamidochrysoidine, Demeclocycline, Doxycycline, Metacycline, Minocycline, Oxytetracycline, Tetracycline, Clofazimine, Dapsone, Capreomycin, Cycloserine, Ethambutol, Ethionamide, Isoniazid, Pyrazinamide, Rifampicin, Rifabutin, Rifapentine, Streptomycin, Arsphenamine, Chloramphenicol, Fosfomycin, Fusidic acid, Metronidazole, Mupirocin, Platensimycin, Quinupristin/Dalfopristin, Thiamphenicol, Tigecycline, Tinidazole, Trimethoprim, itraconazole, posaconazole, ketoconazole, clotrimazole, miconazole, voriconazole, caspofungin, anidulafungin, micafungin, nystatin, amphotericin b, griseofulvin, terbinafine, and flucytosine.

Aspect 13. The method of any one of aspect 1 to 12, wherein the unidentified or unknown microorganism is selected from virus, bacteria, or fungi.

Aspect 14. The method of aspect 13, wherein the unidentified or unknown microorganism is selected from Actinomyces israelii, Bacillus anthracis, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, Yersinia pseudotuberculosis, Absidia corymbifera, Absidia ramose, Achorion gallinae, Actinomadura spp., Ajellomyces dermatididis, Aleurisma brasiliensis, Allersheria boydii, Arthroderma spp., Aspergillus flavus, Aspergillus fumigatu, Basidiobolus spp, Blastomyces spp, Cadophora spp, Candida albicans, Cercospora apii, Chrysosporium spp, Cladosporium spp, Cladothrix asteroids, Coccidioides immitis, Cryptococcus albidus, Cryptococcus gattii, Cryptococcus laurentii, Cryptococcus neoformans, Cunninghamella elegans, Dematium wernecke, Discomyces israelii, Emmonsia spp, Emmonsiella capsulate, Endomyces geotrichum, Entomophthora coronate, Epidermophyton floccosum, Filobasidiella neoformans, Fonsecaea spp., Geotrichum candidum, Glenospora khartoumensis, Gymnoascus gypseus, Haplosporangium parvum, Histoplasma, Histoplasma capsulatum, Hormiscium dermatididis, Hormodendrum spp., Keratinomyces spp, Langeronia soudanense, Leptosphaeria senegalensis, Lichtheimia corymbifera, Lobmyces loboi., Loboa loboi, Lobomycosis, Madurella spp., Malassezia furfur, Micrococcus pelletieri, Microsporum spp, Monilia spp., Mucor spp., Mycobacterium tuberculosis, Nannizzia spp., Neotestudina rosatii, Nocardia spp., Oidium albicans, Oospora lactis, Paracoccidioides brasiliensis, Petriellidium boydii, Phialophora spp., Piedraia hortae, Pityrosporum furfur, Pneumocystis jirovecii (or Pneumocystis carinii), Pullularia gougerotii, Pyrenochaeta romeroi, Rhinosporidium seeberi, Sabouraudites (Microsporum), Sartorya fumigate, Sepedonium, Sporotrichum spp., Stachybotrys, Stachybotrys chartarum, Streptomyce spp., Tinea spp., Torula spp, Trichophyton spp, Trichosporon spp, and Zopfia rosatii.

### EXAMPLES

Electrochemical-based molecular quantification of RNA transcription. An electrochemical-based sensor system (e.g., BsiMax® system) is used for rapid molecular diagnostics directly from human specimens (urine, whole blood, saliva, CSF, etc.) without target purification or nucleic acid amplification. The electrochemical-based sensor is based on sandwich hybridization of capture and detector oligonucleotide probes which target 16S ribosomal RNA (rRNA). The capture probe is anchored to the gold sensor surface, while the detector probe is linked to horseradish peroxidase (HRP). When a substrate such as 3,3′,5,5′-tetramethylbenzidine (TMB) is added to an electrode with capture-target-detector complexes bound to its surface, the substrate is oxidized by HRP and reduced by the bias potential applied onto the working electrode. This redox cycle results in the shuttling of electrons by the substrate from the electrode to the HRP, producing enzymatic signal amplification of current flow in the electrode. The concentration of the RNA target captured on the sensor surface can be quantified by the reduction current measured through the redox reaction between the TMB and HRP with a built-in multi-channel potentiostat in the system.

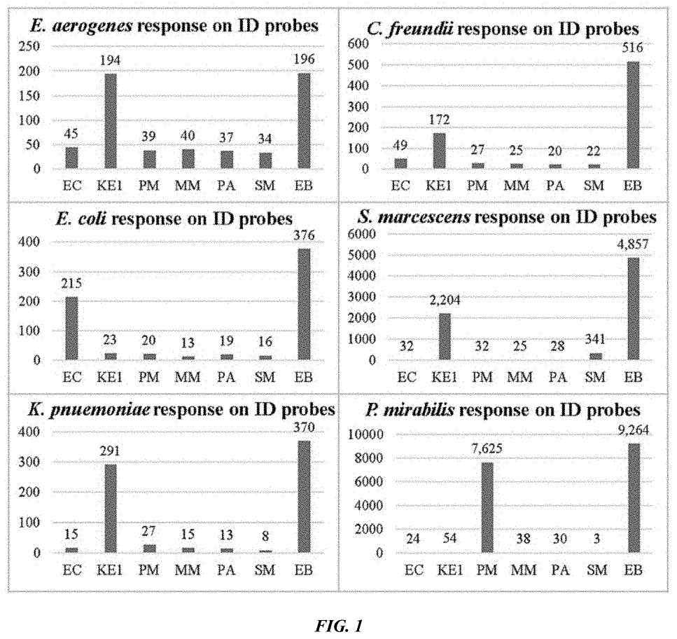

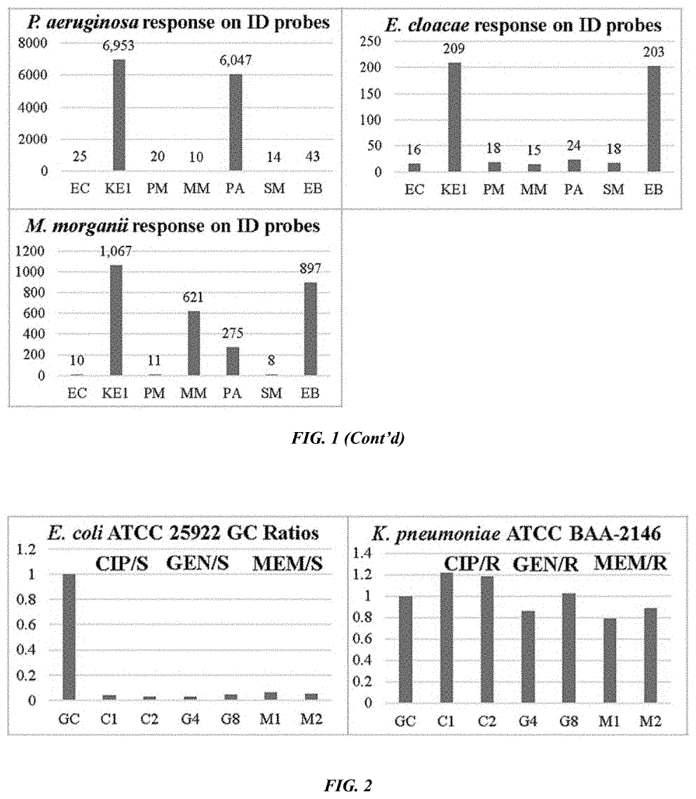

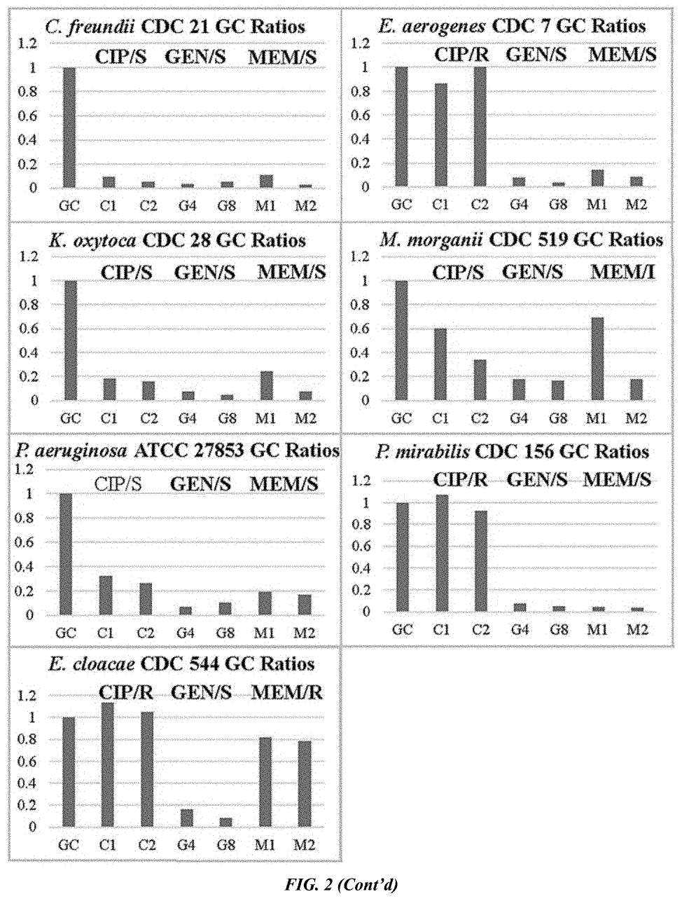

Species-specific pathogen identification directly from clinical specimens. A set of 188 oligonucleotide probe pairs that have been optimized for hybridization at room temperature, 37° C. and 65° C. are used for identification of common pathogens. The current species-specific ID panel targets common pathogens, such as E. coli and P. aeruginosa, while two other probe sets, EB (Enterobacteriaceae) and BU (bacterial universal), serve as internal controls. The bacterial universal (BU) probe pair, which targets a highly conserved region of 16S bacterial rRNA, detects all pathogens tested. The EB probe pair targets the Enterobacteriaceae family of Gram-negative enteric bacteria that constitute the majority of the Gram-negative pathogens. Probe pairs targeting Proteus mirabilis (PM), E. coli (EC), Pseudomonas aeruginosa (PA), Enterococcus spp. (EF), Serratia marcescens (SM), Providencia (PS), Staphylococcus aureus (SA) and Morganella morganii (MM) show specific detection of their respective species. Given the similarity in the 16S rRNA sequences for the Klebsiella, Enterobacter, and Citrobacter spp., the KE probe pair has specificity for Klebsiella pneumoniae, Klebsiella oxytoca, Enterobacter cloacae, Enterobacter aerogenes and Citrobacter freundii. The electrochemical-based sensor system (e.g., BsiMax® system) reports ID and AST directly from whole blood samples, not clinical isolates or positive blood culture bottles. A whole blood sample in BD 367856 Vacutainer EDTA tube can be directly loaded into the electrochemical-based sensor system (e.g., BsiMax® system). Once ID is reported positive, a corresponding phenotypic AST test is carried out automatically. Representative ID and AST results demonstrate species-specific RNA transcription quantification for ID and AST (see FIGS. 1 and 2).

Laboratory automation of molecular analysis platform. The main goal of the robotic system development effort is to combine the advantages of lab automation, rapid molecular analysis, multiplexed detection, genotypic pathogen quantification and phenotypic antibiotic conditions to dramatically improve the sensitivity and specificity of rapid, evidence-based pathogen ID and AST directly from patient samples such as whole blood and urine and to address the assay reproducibility issues due to manual operation. More than 2,000 spiked urine and blood samples were tested for analytical validation and feasibility studies for the CE Marking and FDA 510 (k) clinical testing according to procedures documented under our Good Laboratory Practice (GLP) and ISO 13485 certified quality system. The implementation of robotic automation of the molecular quantification of 16S rRNA transcription as a growth marker on the current BsiMax system comprises sequential protocol steps listed below: (1) Retrieval of ID sensor chips. (2) Loading of sample (blood/urine) tubes. (3-5) Pelleting sample and removal of blood/urine matrix interfering components in the supernatant. (6-7) Viability culture to enrich only viable target pathogens. (8) Concentration of target pathogens in the pellet. (9) Part of the pellet is set aside for subsequent AST if ID reports positive. (10) The remaining ID pellet is lysed and neutralized to stabilize the 16S rRNA content. (11) Resulting lysate of each sample is delivered onto ID chips for hybridization with species-specific oligonucleotide probes immobilized on each sensor. (12) ID chips are transported to the Chip Incubator for stringent hybridization. (13) Stringent wash and dry to remove unbound RNA and matrix components. (14) ID chip is transported onto the ENZ Slider and enzyme reagent is delivered and incubated on each sensor to attach the signaling HRP onto sandwiched DNA/RNA/DNA hybrids. (15) Stringent wash and dry to remove unbound enzyme before the ID chip is transported onto the TMB slider for substrate reagent delivery for enzymatic amplification. (16) ID chip is transported onto the Reader Slider and enzymatic amperometric reading is performed once the Reader Head is fully engaged with the sensor chip. (17) ID results are displayed on GUI and thermal printer. [18] Retrieval of AST sensor chips. [19-20] Delivery of the set aside AST inoculum for each sample into the corresponding AST strip well for the phenotypic AST. [21] Lysing the AST inoculum in each well. [22] Resulting lysate of each well is delivered onto corresponding sensors of AST chips for hybridization. [23] Repeat of (12-17) for stringent hybridization, stringent wash and dry, enzyme delivery, TMB delivery and electrochemical reading for AST reporting on GUI and thermal printer.

Analytical Validation Method, Testing Condition and Data Analysis. Whole blood samples in BD 367884 Vacutainer Lithium Heparin tube or urine/swab samples in BD 364954 Vacutainer® Plus C&S Preservative Tube can be directly loaded into BsiMax® system and the testing menu (AST, checkerboard or HR) can be selected from the GUI touch screen. Since there is no commercially available FDA-cleared system or CLSI reference method to provide AST results directly from specimens without overnight culture or clinical isolates, the acceptance criteria for the analytical validation and clinical testing are the same as conventional AST to demonstrate >95% categorical agreement on 150 contrived specimens (50 blood, 50 urine, and 50 swab) as detailed in CLSI M07 and M100.

Clinical validation of ID, AST and streamlined ID/AST. Feasibility studies have been demonstrated using a molecular-based genotypic-phenotypic-hybrid approach for multiplexed bacterial PID and AST profiling with 92% clinical sensitivity and 97% clinical specificity, yielding a positive predictive value of 99% and a negative predictive value of 81% in a study on 215 clinical urine samples. The reproducibility has been assessed recently in a feasibility study testing 35 strains (CDC AR Bank, ATCC) multiple times over 1 month totaling 143 CIP, 155 GEN, and 152 MEM results in preparation for FDA 510(k) testing. Categorical agreement (CA) and reproducibility of the results in the pilot study are >92% and >95%, respectively.

Strategy for the direct-from-sample AST assay. The change in RNA transcription is among the earliest cellular changes upon exposure to antibiotics, long before phenotypic changes in growth can be observed. Quantifying changes in RNA signatures is therefore a particularly appealing approach for slow-growing organisms. Measuring the RNA response of pathogens to antibiotic exposure directly in the clinical specimens will provide a rapid susceptibility assessment that can be performed in clinical setting. The streamlined ID (green)/AST (yellow) protocols on the current lab automation systems settings. GeneFluidics plans to perform scientific demonstration and diagnostic equivalence studies of direct-from-specimen assays to determine the susceptibility of the infecting strain to at least three classes of antibiotics (quinolones, macrolides, cephalosporins) directly from patient specimens (e.g., whole blood, urine, urethral/vaginal swab). The format of the assay will make it easily adaptable for the testing of different specimen types and different antibiotics on the same robotic lab automation system as they are introduced into the recommended treatment regimen. Additionally, molecular-based tests can be quickly modified to perform three different representative susceptibility assays to address three major root causes of empirical therapy failures; (Aim 1) a direct-from-specimen AST for monoclonal infections resistant to first-line antibiotics, (Aim 2) a direct-from-specimen checkerboard assay for combinational therapy of difficult-to-treat polymicrobial and/or multidrug resistant (MDR) infections, and (Aim 3) a direct-from-specimen heteroresistant (HR) assay for colistin, the last resort therapy option for bacterial infections

Direct-from-specimen AST Lab Automation System Development Method. Due to the complexity of managing multiple assay protocols in Aims 1-3 for multiple specimens with the random-access capability, a Raspberry Pi 4-based Pi-top mini-PC was incorporated into the system to boost up the operating capacity and speed needed for signal processing (Broadcom BCM2711 SoC with 4× Cortex-A72 cores clocked to 1.5 GHz, 4 GB LPDDR4, native GbE and dual 4K-ready micro-HDMI ports) and a serial port expander to increase the number of controlled components from 32 to 160. Specifications and protocols needed to implement the firmware are evaluated and implemented. Acceptance criteria are evaluated with contrived and remnant clinical samples along with gold standard methods at GeneFluidics and NYPQ to finalize a reporting algorithm and assay parameters according to the triple-response-curve signature and categorical classification correlation.

Due to the complexity of integrating multiple protocols for various specimen types with different timing and signature libraries, the system needs to plan out a testing plan, choosing which samples to test first, in what order, and within a given specimen delay time. If insufficient sample volume or inconsistent specimen type prevents the robot from initiating or completing an assay protocol, it can choose to drop that sample, sound an alarm or reconfigure the protocol to recover from a failure while processing other clinical samples simultaneously. Since the whole blood matrix is more complex with a higher viscosity than urine and swab samples, the assay protocol can be optimized after the initial demonstration (see FIG. 5). A more powerful core (Pi-top [4]), however, is needed to provide the high level of intelligence in the current autonomous robot system so that it can coordinate effectively with various specimen types and procedures listed in Table 2. The Amazon real-time operating system (RTOS) self-scheduling capability of the cognitive core requires specimen-specific automated preanalytical protocols and associated mechanical modules. Categorical agreement is validated independently for each specimen type with 150 contrived and 450 remnant clinical specimens.

TABLE 2
Generations of robotic systems for coordinating various specimen types and procedures

| | Hand-eye | Cognitive | Specimen/ | Matrix | ID/AST |
|---|---|---|---|---|---|
| Generation | coordination | Control | assay | Management | panel |
| 1rst gen. | None. | None. Teensy | Selected from a | Predetermined | Gram-negative |
| | Warning in the | 3.5 on a fixed | set menu on | automated | |
| | user manual to | protocol | GUI | procedures for | |
| | prevent | | | blood/urine | |
| | failure. | | | | |
| 2^{nd} gen. | None. | None. | Selected from a | Predetermined | Gram- |
| | Warning in the | | set menu on | procedures for | negative/positive |
| | user manual to | | GUI | blood and | |
| | prevent | | | urine | |
| | failure. | | | | |
| Current | IS2000M | Manages up | Scans the | Manipulates | S. aureus, |
| gen. | vision system | to 160 | barcode on each | samples in | S. epidermidis, |
| | to coordinate | components, | collection tube | real-time | CoNS, |
| | and process | sensory | to determine the | with image | Enterococcus, |
| | the | inputs, image | assay protocol | recognition | E. coli, |
| | information | processing | and record | | E. cloacae, |
| | received | and executive | patient ID | | K. pneumoniae, |
| | | motion with | | | P. aeruginosa, |
| | | 4GB RAM | | | S. marcescens, |
| | | and ARM | | | P. mirabilis, |
| | | Cortex-73- | | | A. baumannii, |
| | | based | | | MRSA, VRE |
| | | processor | | | |

Direct-from-specimen AST Clinical Feasibility Validation Workflow and Protocols. The clinical remnant specimens for this study will use previously collected specimens, such as archived specimens or leftover specimens (also called surplus specimens). Remnant specimens are the remainders of specimens collected for routine diagnostic testing that would otherwise have been discarded, or specimens that were previously collected for other research purposes (e.g., basic research studies, pharmaceutical clinical trials, previous IVD medical device clinical performance studies). De-identification and data analysis are performed by medical technologists. When a culture (blood, urine or vaginal/rectal swab) is sent for laboratory evaluation, the medical technologist will start tracing specimen routing and set aside all remnant specimens. If antibiotic treatment is reported as part of standard care, then the remnant specimens are de-identified, coded and sent for analysis on BsiMax in CLIA laboratories located in the same building as the clinical microbiology lab. The current IRB are revised to reflect the change in specimen types and collection procedures. Inclusion Criteria: (1) Clinical samples collected within 72 hours, (2) From patients between 1 and 18 years of age, (3) Reliable specimen without any known or verified contaminations, (4) Indication for antibiotic treatment with ciprofloxacin, gentamicin or meropenem. Exclusion Criteria: (1) Patients previously infected or colonized with multidrug-resistant pathogens and moribund patients. (2) Positive culture for infections at NYPQ within prior 7 days (if known at the time of collection). (3) Available sample volume to be at least 4 mL for urine/swab and 2 mL for blood for testing and re-plating if there is a discrepancy.

Direct-from-specimen AST Analytical Validation Protocol: (1) The user loads the specimen collection tube into the system, which then (2) scans the bar code to determine specimen type. (3) Image recognition function determines the volume of the specimen. (4) The system pellets the sample by spinning down then removing supernatant once for urine and swab samples and twice for blood. (5) The system inoculates the pellet with culture media into 1× inoculum, then (6) aliquots and dilutes into two additional inoculum concentrations with dilution factors (Table 4). (7) The 3 inoculums are added into 3 stripwells for antibiotic exposure inside the system for incubation time (Table 3) and remaining suspension is added to glycerol for archival or re-testing. (8) The samples are lysed then delivered to sensors for hybridization, followed by (9) a stringency wash. The system incubates enzyme, then performs electrochemical reading and (10) AST reporting.

TABLE 3
Parameter optimization approaches for direct-from-specimen AST

| | Conditions to be | |
|---|---|---|
| Conditions | optimized | Considerations |
| Inoculum | 1x, 0.1x, 0.01x | Identify 3 inoculum concentrations (undiluted and 2 |
| dilution factors | 1x, 0.2x, 0.04x, | diluted) to assess the maximum separation of GC ratio |
| | 1x, 0.5x, 0.25x | response curves between resistant and susceptible |
| | 1x, 0.3x, 0.1x | strains |
| Antibiotic | 15, 60, 120 min for | Identify min. antibiotic exposure time to distinguish |
| exposure time | urine/swab | GC response curves between the slowest growing |
| | 2, 3, 4 and 5 hours | resistant and susceptible PA strains with slowest |
| | for blood | reacting antibiotic MEM |
| Antibiotic | Seven 2-folds above | The antibiotic response can be amplified by a range of |
| concentration | S breakpoint | higher antibiotic 2-fold dilutions from around the |
| | Seven 2-folds above | intermediate (I) breakpoint to above S and R |
| | R breakpoint | breakpoints |

Direct-from-specimen AST Clinical Feasibility Validation Data Analysis. The first level of analysis is qualitative, whereby all triple-response-curve signatures are compared to the corresponding antibiotic susceptibility profile (R for resistant, I for intermediate or S for susceptible) for identified pathogen with susceptibility results from the clinical microbiology lab, and any direct-from-specimen AST found to be misclassified (i.e., all GC ratios close to 1.0 for a susceptible strain) are identified and retested with both BsiMax and disk diffusion reference methods. All samples with signal levels lower than the assay cutoff are reported “GC fail,” and the time-to-positivity of the original specimen culture are located and correlated to determine the ability to test low abundance pathogen levels. Categorical agreements are calculated for each specimen type. As a second level of analysis for MIC reporting only, the out-of-range MIC values (i.e., 2-fold above or below the one from the clinical microbiology lab) are retested and compared to the microdilution reference method. Essential agreements are calculated for each specimen type. For any discrepancies in AST reporting, the remaining clinical samples are tested with CLSI reference methods and the results are analyzed.

Methodology for Direct-from-specimen determination of combinational therapy for MDR infections. Due to the large volume needed to load the 96-well plate, BD 367880 Vacutainer Lithium Heparin tubes are used for whole blood, and BD 364979 Vacutainer® Urinalysis tubes are used for urine/swab samples. A modified swing bucket is fitted into the current centrifugation module with a spring-loaded clamper, the swing bucket accommodates 13 mm to 16 mm diameter of collection tubes (4 mL to 10 mL capacity). The analytical validation and clinical feasibility study are the same as described above to assess the inhibited growth of 60 contrived specimens (20 blood, 20 urine, and 20 swab) with CDC AR Bank strains and 90 (30 blood, 30 urine and 30 swab) remnant clinical specimens. The method identifies a combinational therapy exhibiting the maximum therapeutic effect during emergencies. The inhibited growth of all 96 antibiotic combination conditions are compared to the CLSI reference microdilution method using the same 96-well plate after 18-24 hours of incubation. The acceptance criteria for the analytical validation and clinical testing are ≥90% agreement with the ten antibiotic combinations with most inhibited growth (i.e., highest therapeutic conditions).

Protocol for direct-from-specimen determination of combinational therapy for subjects with MDR: (1) The user loads the specimen collection tube into the system, which then (2) scans the bar code to determine specimen type. (3) Image recognition function determines the volume of the specimen. (4) The system pellets the sample by spinning down then removing supernatant once for urine and swab samples and twice for blood. (5) The system inoculates the pellet with culture media into 1× inoculum. (6) The system will deliver 10 μL of the 1× inoculum into each well of the 96-well plate for an appropriate antibiotic exposure time. (7) Lysing reagents are added to each well by the system then the lysate is delivered to associated sensor for hybridization. (8) A stringency wash, enzyme incubation, and electrochemical reading as shown in FIG. 3 Steps 13-16 is performed. (9) The inhibited growth is then reported.

Direct-from-specimen determination of combinational therapy for subjects with MDR Data Analysis. The analytical validation and clinical feasibility study are the same as described above, but the criteria to assess the 10 antibiotic combinations out of 96 conditions tested with most inhibited growth (i.e., highest therapeutic conditions) for 60 contrived specimens (20 blood, 20 urine, and 20 swab) with CDC AR Bank strains and 90 (30 blood, 30 urine and 30 swab) remnant clinical specimens from NYPQ with a ≥90.0% agreement with microdilution methods. The inhibited growth is quantified by growth control (GC) ratios as shown in FIGS. 2, 5C, 6. Criteria for re-examination and resolution: any discrepancies are tabulated and recorded in a daily log. For any discrepancies in combination reporting, the remaining clinical sample is tested with E-test and CLSI macrodilution methods and the results are analyzed.

Direct-from-specimen of HR approach. HR is a poorly characterized phenomenon and the lack of consensus-based standards further complicates the study of antibiotic resistance. In addition, its clinical relevance is uncertain due to a lack of a standardized procedure to be implemented into the current workflow in clinical microbiology laboratories. A preliminary accelerated colistin AST was demonstrated with only 2 hours of drug-bug exposure time in FIG. 8. Colistin (CST) susceptible (SCST) strains such as CDC Enterobacter cloacae (EbC) 545, MIC≤0.25 μg/mL and Klebsiella pneumoniae (KP) 138, MIC=2 μg/mL exhibit clear inhibited growth (FIG. 8A) with just 2 hours of CST exposure in a customized stripwell while the growth of CDC KP 522 and KP 525 (both MIC>8 μg/mL) were not affected by the tested colistin concentration range (0.25-8 μg/mL). In addition, the MIC of CDC Proteus mirabilis (PM) 29 was listed as >4 μg/mL, but it was the least susceptible to the colistin than all other strains tested, a result in agreement with the fact that P. mirabilis is intrinsically resistant to colistin. FIG. 8C is the HR testing on the system.

Direct-from-specimen of HR methods and protocols. The initial effort of colistin HR detection will focus on agreement with the PAP gold standard method using contrived specimens with CDC RCST and SCST strains to demonstrate the ability of detecting low frequency (10⁻⁵-10⁻⁷) of HR population under selected colistin conditions as demonstrated in FIG. 8 using direct-from-specimen AST protocols instead of starting from isolates. CDC MIC values are verified with agar dilution testing. The validated direct-from-specimen HR will mainly rely on contrived samples with blood, urine and swab specimens. Specifically, 100 μL of a characterized strain such as CDC 349 E. coli (colistin MIC: 2-4 μg/mL) at 10⁷ CFU/mL is used as inoculum on colistin plates with 0, 1, 2, 4, 8 μg/mL. The current BsiMax AST system uses higher inoculum concentration for an accelerated AST in order to achieve >95% categorical agreement with only two hours of incubation instead of 16-24 hours, and the drug/bug ratio was reviewed and accepted by FDA. As shown in FIG. 8F, the elimination of dominant susceptible (d-S) was insignificant compared to exponential growth of minority resistant (m-R) at 8 hours (T8). Acceptance criteria—A direct-from-specimen HR assay to detect m-R/d-S at 1/10⁵ to 1/10⁸ (R/S) ratio in 8 hours.

Other embodiments, combinations and modifications of this invention will occur readily to those of ordinary skill in the art in view of these teachings. Therefore, this invention is to be limited only by the following claims, which include all such embodiments and modifications when viewed in conjunction with the above specification and accompanying drawings.

1. A method to assess the antimicrobial susceptibility profile of unidentified and/or unknown pathogens to a panel of antimicrobials directly from an unprocessed sample without the use of clinical isolates, comprising steps (i) or (ii); and (iii):
(i) measuring from the unprocessed sample, a change in the level of a growth marker for the unidentified and/or unknown pathogens when the pathogens are exposed to the panel of antimicrobials; or
(ii) measuring from the unprocessed sample, a change in the level of a growth marker for the unidentified and/or unknown pathogens when the unprocessed sample with unidentified and/or unknown pathogens are diluted to different dilution levels and exposed to the panel of antimicrobials at various antimicrobial/pathogen ratios; and
(iii) comparing the change in the level of the growth marker for unidentified and/or unknown pathogen from steps (i) or (ii) to the level of the growth marker when the unidentified and/or unknown pathogens exposed to a growth control (GC) condition that lacks antimicrobials,
wherein the susceptibility of the unidentified and/or unknown pathogens to the panel of antimicrobials is determined by a change in the level of the growth marker in comparison to the level of the growth marker when the unidentified and/or unknown pathogens are exposed to the growth control condition that lacks antimicrobials.

2. The method as recited in claim 1, wherein the growth marker is selected from nucleic acids, proteins, and phenotypic characteristics.

3. The method as recited in claim 2, wherein the growth marker is RNA, and wherein the change of RNA content is measured using one or more molecular analysis assays that utilize pathogen species-specific quantification, pathogen class-specific quantification, and/or universal quantification.

4. The method as recited in claim 3, wherein the pathogen species specific quantification includes specific quantification of Escherichia coli, Klebsiella pneumoniae, and/or methicillin-resistant Staphylococcus aureus (MRSA); and wherein the pathogen class-specific quantification includes specific quantification of Enterobacteriaceae, Gram-negative bacteria, and/or Gram-positive bacteria.

5. The method as recited in claim 2, wherein the growth marker is RNA, and wherein the change of RNA content is measured using one or more molecular analysis assays that utilize enzymatic signal amplification with electrochemical sensors.

6. The method as recited in claim 1, wherein the pathogens in the unprocessed sample are exposed to the panel of antimicrobials by using a macrodilution assay, a microdilution assay, by agar plating, or by culturing in growth media culture.

7. The method as recited in claim 6, wherein:
a defined set of concentrations for the panel of antimicrobials are used; or
a range of concentrations for the panel of antimicrobials are used; or
various antimicrobial-to-pathogen ratios are used; or
the pathogens are exposed to the panel of antimicrobials using different exposure times.

8. The method as recited in claim 7, wherein the defined set of concentrations for the panel of antimicrobials includes concentrations that provide for susceptible, intermediate and/or resistance breakpoints.

9. The method as recited in claim 7, wherein the defined set of concentrations for the panel of antimicrobials includes concentrations that provide for a 2-fold increase or decrease from susceptible, intermediate and/or resistance breakpoints.

10. The method as recited in claim 7, wherein the defined set of concentrations for the panel of antimicrobials includes concentrations that provide for a more than 2-fold increase or decrease from susceptible, intermediate and/or resistance breakpoints.

11. The method as recited in claim 7, wherein the defined set of concentrations for the panel of antimicrobials includes from 2-12 different concentrations for the panel of antimicrobials.

12. The method as recited in claim 1, wherein pathogens culture or diluted to different dilution or microbial levels is selected from viable pathogens cultured or diluted to a set number of different dilution levels, viable pathogens cultured or diluted within a range of dilution or microbial levels, and pathogens concentrated to different levels.

13. The method as recited in claim 12, wherein viable pathogens culture or diluted to different dilution or microbial levels includes pathogens diluted to 1×, 0.5×, 0.3×, 0.1×, 0.01×, 0.001×, 0.0001× and/or 0.00001×.

14. The method as recited in claim 12, wherein pathogens concentrated to different levels is by concentrating for different centrifugation times, by concentrating using different centrifugation forces and/or by taking up the pathogen pellet in different volumes.

15. The method as recited in claim 1, wherein the panel of antimicrobials includes 2, 3, 4, 5, 6, 7, 8, 9 or 10 antimicrobials.

16. The method as recited in claim 1, wherein the panel of antimicrobials comprises ciprofloxacin, gentamicin, and/or meropenem.

17. The method as recited in claim 1, wherein the method is used to assess in an unprocessed sample:
(i) the antimicrobial susceptibility profile of a pathogen in a mono-microbial sample; or
(ii) the antimicrobial susceptibility profile of multiple types of pathogens in a poly-microbial sample;
(iii) the antimicrobial susceptibility profile of a multiple-drug-resistant pathogen in a mono-microbial sample; or
(iv) the antimicrobial susceptibility profile of each multiple-drug-resistant pathogens, in a poly-microbial sample.

18. The method as recited in claim 17, wherein a defined set of concentrations for the panel of antimicrobials are used, and wherein the set of concentrations for the panel of antimicrobials provides for 13-1024 different concentrations of the antimicrobials from the panel of antimicrobials in order to carry out the antimicrobial susceptibility profile analysis of pathogens defined in (i), (ii), (iii) or (iv), and wherein each of the 13-1024 different concentrations contains only one type of antimicrobial from the panel of antimicrobials.

19. The method as recited in claim 17, wherein a defined set of concentrations for the panel of antimicrobials are used, and wherein the set of concentrations for the panel of antimicrobials provides for 13-1024 different concentrations of the antimicrobials from the panel of antimicrobials in order to carry out the antimicrobial susceptibility profile analysis of pathogens defined in (i), (ii), (iii) or (iv), and wherein each of the 13-1024 different concentrations contains one type or multiple types of antimicrobial(s) from the panel of antimicrobials.

20. The method of claim 1, wherein the method further comprises steps (iv) and/or (v):
(iv) measuring the growth of the unidentified and/or unknown pathogens within a pre-determined viability culture time after removing matrix interference components; and/or
(v) measuring the growth of the unidentified and/or unknown pathogens within a pre-determined viability culture time after concentrating the pathogens in the unprocessed sample.

21. The method as recited in claim 20, wherein the pre-determined viability culture time is selected from 5 min, 30 min, 1 hour, 2 hours, 3 hours, 6 hours, 12 hours, and 18 hours.

22. The method as recited in claim 20, wherein the unidentified and/or unknown pathogens of step (iv) and/or (v) are concentrated in the unprocessed sample by centrifugating the unprocessed sample to pellet the unidentified and/or unknown pathogens and then removing supernatant.

23. The method as recited in claim 20, wherein for step (iv) and/or (v) the unidentified and/or unknown pathogens are indicated as being antimicrobial susceptibility profile growth of the unidentified and/or unknown pathogens is classified as no observed growth, limited growth, minimum growth, and relatively low growth.

24. The method as recited in claim 17, wherein the antimicrobial susceptibility profile includes but is not limited to homogeneous microbial populations, heterogeneous microbial populations, pseudo-homogeneous microbial populations and pseudo-heterogeneous microbial populations.

25. The method as recited in claim 17, wherein the antimicrobial susceptibility profile includes but is not limited to the case where the majority of a heterogeneous microbial population is susceptible to a given antibiotic and a minority of the population is resistant, so the growth of the minority population will only be observed after the inhibited growth of the susceptible majority is observed.

26. The method as recited in claim 1, wherein the method is fully automated by the use of a robotic handling system to carry out the method steps.
